(19) United States
(12) Patent Application Publication
(10) Pub. No.: US 2015/0297189 A1
(43) Pub. Date: Oct. 22, 2015

(54) ULTRASOUND DIAGNOSTIC APPARATUS

(71) Applicant: HITACHI ALOKA MEDICAL, LTD., Mitaka-shi,Tokyo (JP)

(72) Inventors: Yuya Shishido, Mitaka- shi (JP); Masaru Murashita, Mitaka- shi (JP); Toshinori Maeda, Mitaka- shi (JP)

(73) Assignee: HITACHI ALOKA MEDICAL, LTD., Mitaka-shi, Tokyo (JP)

(21) Appl. No.: 14/647,024

(22) PCT Filed: Nov. 27, 2013
(86) PCT No.: PCT/JP2013/081963

§ 371 Date: May 22, 2015

(30) Foreign Application Priority Data
Nov. 27, 2012 (JP) 2012-258309

(51) Int. Cl. A61B 8/08; A61B 8/00; A61B 8/14; G01S 15/89; G01S 7/52

(52) U.S. Cl. CPC A61B 8/5269; G01S 15/8977; G01S 7/52034; A61B 8/14; A61B 8/4444; A61B 8/461; A61B 8/54; A61B 8/5207

(57) ABSTRACT

A densification processing unit (20) densifies image data composed of a plurality of pieces of line data corresponding to a plurality of ultrasound beams obtained by scanning with an ultrasonic beam (a transmission beam and a reception beam). The densification processing unit (20) densifies the image data by compensating for density of scanning direction data arranged at a low density along the scanning direction of the ultrasonic beam on the basis of depth direction data arranged at a high density along the depth direction of the ultrasonic beam within the imaging data.

### TECHNICAL FIELD

The present invention relates to an ultrasound diagnostic apparatus, and more particularly to a technique of increasing the density of an ultrasound image.

### BACKGROUND ART

Use of an ultrasound diagnostic apparatus enables real-time capturing of a moving image of a tissue in motion, for example, for diagnosis. In recent years, ultrasound diagnostic apparatuses are extremely important medical devices especially in diagnosis and treatment of the heart and other organs.

It is desirable not only for the purpose of diagnosing the heart but also for other purposes that an ultrasound image obtained by an ultrasound diagnostic apparatus has excellent image quality. A technique of increasing the density of an ultrasound image is being proposed as a specific countermeasure for enhancing the quality of an ultrasound image.

Patent Document 1, for example, describes a technique of performing pattern matching processing, for each pixel of interest on the previous frame, between the previous frame and the current frame, and, based on the original group of pixels forming the current frame and the additional group of pixels defined, for each pixel of interest, by the pattern matching processing, increasing the density of the current frame.

Patent Document 2 describes a technique of defining a first pixel array, a second pixel array, and a third pixel array in a frame, executing pattern matching processing, for each pixel of interest on the first pixel array, between the first pixel array and the second pixel array to calculate a mapping address on the second pixel array for the pixel of interest, further executing pattern matching processing, for each pixel of interest on the third pixel array, between the third pixel array and the second pixel array to calculate a mapping address on the second pixel array for the pixel of interest, and, with the use of pixel values and the mapping addresses of the plurality of pixels of interest, increasing the density of the second pixel array.

It is possible to increase the density of a low-density image obtained at a high frame rate using the techniques described in Patent Document 1 and Patent Document 2.

Among methods of scanning an ultrasound beam, in the sector scanning and the convex scanning, an ultrasound beam is scanned radially or in a sector shape about a center which is located on the probe side. Accordingly, the interval of the ultrasound beams is greater in a deep portion distant from the probe than in a shallow portion near the probe. It is therefore desired that, even if the interval of the ultrasound beams is wide as described above, the density can be increased so as to fill the interval.

### CITATION LIST

### Patent Literature

[Patent Document 1] JP-2012-105750 A

[Patent Document 2] JP-2012-105751 A

### SUMMARY OF INVENTION

### Technical Problem

In view of the background art described above, the inventor of the present invention has repeated research and development concerning an improved technique of increasing the density of an ultrasound image. In particular, the present inventor has noted a technique of increasing the density of an ultrasound image based on a principle which is different from those of the epoch-making techniques described in Patent Document 1 and Patent Document 2.

The present invention has been conceived during the course of the research and development described above and is aimed at providing an improved technique of increasing the density of an ultrasound image by using a density-based relationship between the scanning direction and the depth direction of the ultrasound beam.

### Solution to Problem

In order to attain the above object, an ultrasound diagnostic apparatus in accordance with a preferable aspect includes a probe configured to transmit and receive ultrasound, a transmitter/receiver unit configured to control the probe to scan an ultrasound beam, a density-increasing processing unit configured to increase a density of imaging data obtained by scanning the ultrasound beam, and a display processing unit configured to form a display image based on the imaging data having an increased density, and the density-increasing processing unit, based on depth direction data arranged at a high density along a depth direction of the ultrasound beam within the imaging data, augments a density of scanning direction data arranged at a low density in a scanning direction of the ultrasound beam, thereby increasing the density of the imaging data.

In the above structure, various types of probes which transmit and receive ultrasound, including a convex scanner type, a sector scanner type, and a linear scanner type, for example, may be used in accordance with the type of diagnostic use. The density increasing which is implemented by the above structure is particularly preferably realized by a combination of a convex scanner and a sector scanner. Also, either a probe for a two-dimensional tomographic image or a probe for a three-dimensional image may be used. While a two-dimensional tomographic image (B mode image) is a preferable example image to be subjected to density increasing, a three-dimensional image, a Doppler image, or an elastography image may also be adopted. The imaging data refers to data which is used for forming an image, and is line data obtained along the ultrasound beams which are scanned, for example.

In the depth direction of the ultrasound beam, as it is possible to sequentially obtain a received signal of ultrasound from the shallow portion (on the side close to the probe) through the deep portion (on the side distant from the probe), depth direction data arranged at a relatively high density can be obtained. It is, for example, possible to obtain several thousand line data units along a single ultrasound beam, and the several thousand line data units may be used as they are or several hundred line data units obtained by resampling (decimation) the several thousand line data units may be used. A plurality of ultrasound beams are sequentially formed by scanning the ultrasound beams, while, for example, shifting the position (angle) of the ultrasound beam stepwise along the scanning direction. In the case of a general two-dimensional B-mode image, the number of ultrasound beams used for obtaining one image (one frame) is about 100, for example. In order to increase the frame rate, for example, it is necessary to further reduce the number of ultrasound beams. Accordingly, the scanning direction data is arranged in the scanning direction of ultrasound beam at a relatively low density. As described above, the data which is obtained differs in density between the scanning direction and the depth direction of the ultrasound beam.

The above apparatus can realize density increasing of an ultrasound image by using a density-based relationship between the scanning direction and the depth direction of the ultrasound beams. More specifically, by augmenting the density of the scanning direction data arranged at a low density along the scanning direction of the ultrasound beam based on the depth direction data arranged at a high density along the depth direction of the ultrasound beam, the density of the imaging data is increased.

In a preferable specific example, the density-increasing processing unit places a template corresponding to the scanning direction of the ultrasound beam within the imaging data and moves a kernel corresponding to the depth direction of the ultrasound beam for searching for a kernel that matches the template, thereby augmenting a density of the scanning direction data that belongs to the template by using the depth direction data that belongs to the kernel which has been found.

In the above structure, the template is preferably set so as to enclose the scanning direction data, for example, and may have a one-dimensional shape or a two-dimensional shape. If the imaging data is three-dimensional data, a template having a three-dimensional shape may be used. The kernel is preferably set so as to enclose the depth direction data, for example, and may have a one-dimensional shape or a two-dimensional shape. If the imaging data is three-dimensional data, a kernel having a three-dimensional shape may be used. A template and a kernel preferably have identical shapes.

In a preferable specific example, the density-increasing processing unit searches for a kernel that matches the template by pattern matching between the scanning direction data that belongs to the template and the depth direction data that belongs to the kernel.

In a preferable specific example, the density-increasing processing unit searches for a kernel that matches the template by pattern matching based on a degree of similarity between the scanning direction data within the template and the depth direction data to be selected from the kernel at a data interval of the scanning direction data.

In the above structure, the degree of similarity refers to an indicator for evaluating a similarity level, and may be an indicator which indicates a smaller value as the similarity is greater (more similar) or an indicator which indicates a greater value as the similarity is greater. While, as the indicator for evaluating the similarity level, a sum of squares concerning a difference between data items to be compared with each other and a sum of absolute values concerning a difference between data items to be compared with each other, for example, are preferable, various other known operation methods may be used.

In a preferable specific example, the density-increasing processing unit inserts the density-increasing data obtained based on the depth direction data within the kernel that matches the template into a gap of the scanning direction data within the template, thereby increasing the density of the imaging data.

In a preferable specific example, the density-increasing processing unit assumes a location where the degree of similarity is the best in the gap of the scanning direction data within the template, based on a spatial distribution of the degree of similarity obtained for the search for the kernel that matches the template, and inserts the density-increasing data in the location which is assumed.

In a preferable specific example, the density-increasing processing unit searches for a plurality of candidate kernels that match the template by pattern matching, and, based on a distance between each of the candidate kernels and the template, selects a kernel that matches the template from among the plurality of candidate kernels.

In a preferable specific example, the density-increasing processing unit selects a plurality of kernels that match the template, and, based on the depth direction data obtained from the plurality of kernels, obtains the density-increasing data to be inserted in a gap of the scanning direction data within the template.

In a preferable specific example, the density-increasing processing unit obtains the density-increasing data based on the depth direction data obtained from the plurality of kernels that match the template and a distance between each of the kernels and the template.

In a preferable specific example, the density-increasing processing unit sets the template and the kernel so as to be of identical size in a real space.

In a preferable specific example, the density-increasing processing unit, for increasing the density of the imaging data obtained by scanning the ultrasound beam radially or in a sector shape, increases a size of the template in a real space as the depth of a location where the template is to be placed within the imaging data is greater.

In a preferable specific example, the density-increasing processing unit, for searching for a kernel that matches the template by pattern matching based on a degree of similarity between the scanning direction data within the template and the depth direction data to be selected at a data interval of the scanning direction data from within the kernel, increases the data interval of the depth direction data to be selected from within the kernel as the depth of the location of the template is greater.

In a preferable specific example, the density-increasing processing unit places templates at a plurality of different locations within the imaging data and searches for a kernel that matches the template at each of the locations, thereby increasing the density of the scanning direction data that belongs to the templates at the plurality of locations.

In a preferable specific example, the density-increasing processing unit sets the number of the scanning direction data that belong to the template to a fixed value at each of the plurality of locations within the imaging data.

In a preferable specific example, the density-increasing processing unit, for placing a template at a plurality of different locations within the imaging data and searching for a kernel that matches the template at each of the locations, thereby increasing the density of the scanning direction data that belongs to the template at the plurality of locations, sets a size of the template in a real space to a fixed size at each of the plurality of locations within the imaging data.

### Advantageous Effects of Invention

The present invention can realize density-increasing of an ultrasound image by using a density-based relationship between the scanning direction and the depth direction of an ultrasound beam. According to a preferable embodiment of the present invention, for example, the density of the scanning direction data arranged at a low density along the scanning direction of the ultrasound beam is augmented based on the depth direction data arranged at a high density along the depth direction of the ultrasound beam, thereby increasing the density of the imaging data

### BRIEF DESCRIPTION OF DRAWINGS

FIG. 1 is a block diagram illustrating the overall structure of an ultrasound diagnostic apparatus according to a preferable embodiment of the present invention.

FIG. 2 is a view illustrating specific example imaging data obtained by scanning an ultrasound beam.

FIG. 3 is a view illustrating specific example search using a template and a kernel.

FIG. 4 is a view for explaining a data interval within a real space.

FIG. 5 is a view illustrating specific example density increasing by using density-increasing data.

FIG. 6 is a view illustrating specific example density-increased imaging data.

FIG. 7 is a view illustrating example insertion of density-increasing data in consideration of a distance.

FIG. 8 is a view illustrating example insertion of density-increasing data using a plurality of kernels K.

FIG. 9 is a view illustrating specific example assumption concerning the insertion location of the density-increasing data.

FIG. 10 is a view illustrating example insertion of density-increasing data into a corresponding point location.

FIG. 11 is a view illustrating specific example density increasing by using corresponding point locations.

FIG. 12 is a view illustrating imaging data having been subjected to density increasing by using the corresponding point locations.

FIG. 13 is a view illustrating specific example interpolation processing performed in the digital scan converter.

FIG. 14 is a flowchart showing a summary of processing performed by the ultrasound diagnostic apparatus illustrated in FIG. 1.

FIG. 15 is a view illustrating a specific example low-density image.

FIG. 16 is a view illustrating a specific example 1 of a high-density image.

FIG. 17 is a view illustrating a specific example 2 of a high-density image.

FIG. 18 is a view illustrating a specific example 3 of a high-density image.

FIG. 19 is a view illustrating a specific example 4 of a high-density image.

FIG. 20 is a view for explaining various processing applied to line data.

FIG. 21 is a view for explaining filter processing in the depth direction applied to the density-increased imaging data.

FIG. 22 is a view illustrating a specific example of pattern matching.

FIG. 23 is a view for explaining modification example processing performed by the density-increasing processing unit.

FIG. 24 is a view for explaining a modification example with an enlarged search area.

### DESCRIPTION OF EMBODIMENTS

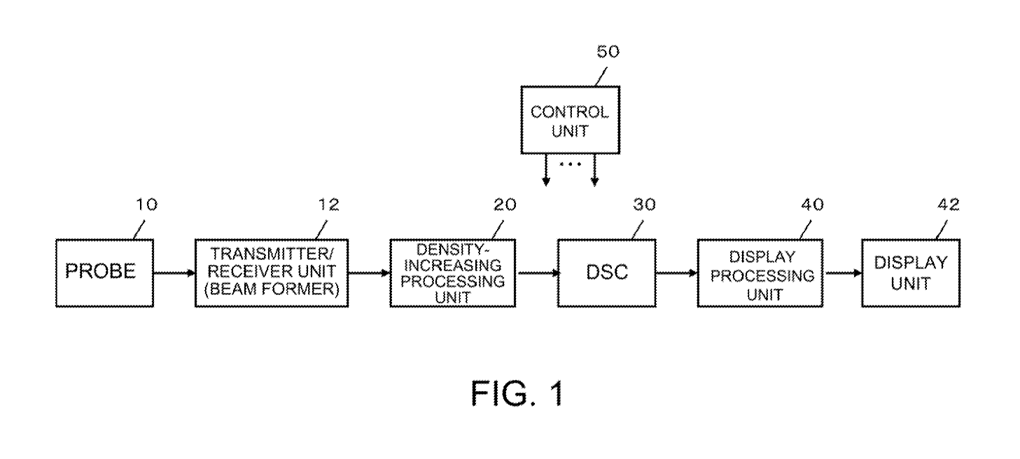

FIG. 1 is a block diagram illustrating the overall structure of an ultrasound diagnostic apparatus according to a preferable embodiment of the present invention. A probe 10 is an ultrasound probe which transmits and receives ultrasound. In accordance with different types of diagnosis, various types of the probe 10 can be used, including a convex scanning type, a sector scanner type, a linear scanner type, a probe for a two-dimensional image (tomographic image), a probe for a three-dimensional image, and other types.

A transmitter/receiver unit 12 controls transmission concerning a plurality of transducer elements included in the probe 10 to form a transmitting beam, and scans the transmitting beam within a diagnosis region. The transmitter/receiver unit 12 also applies phase alignment and summation processing and other processing on a plurality of received signals obtained from the plurality of transducer elements to form a received beam, and collects a received beam signal from the whole region within the diagnosis region. In other words, the transmitter/receiver unit 12 has a function of a beam former. The received beam signals (RF signals) which are collected are subjected to received signal processing including detection processing. Consequently, line data obtained, for each received beam, along the received beam are transmitted to a density-increasing processing unit 20.

The density-increasing processing unit 20 increases the density of imaging data formed of a plurality of line data units corresponding to a plurality of ultrasound beams obtained by scanning the ultrasound beams (transmitting beam and received beam). The density-increasing processing unit 20 specifically increases the density of the imaging data by augmenting the density of scanning direction data arranged at a low density along the scanning direction of the ultrasound beam based on depth data arranged at a high density along the depth direction of the ultrasound beam in the imaging data. The specific processing performed by the density-increasing processing unit 20 will be described in detail below.

A digital scan converter (DSC) 30 applies coordinate transformation processing, frame rate adjustment processing, and other processing to the imaging data having the density increased in the density-increasing processing unit 20; that is, a plurality of density-increased line data units. The digital scan converter 30 obtains image data corresponding to a display coordinate system from a plurality of line data units obtained in a scanning coordinate system corresponding to the scanning of an ultrasound beam, by using coordinate transformation processing, interpolation processing, and other processing. The digital scan converter 30 also converts the plurality of line data units obtained at a frame rate of the scanning coordinate system to the image data at a frame rate of the display coordinate system.

A display processing unit 40 synthesizes the image data obtained by the digital scan converter 30 with graphic data and the like to form a display image, which is displayed on a display unit 42 implemented, for example, by a liquid crystal display. Finally, a control unit 50 controls the entire ultrasound diagnostic apparatus illustrated in FIG. 1.

Among the elements (respective function blocks) illustrated in FIG. 1, the transmitter/receiver unit 12, the density-increasing processing unit 20, the DSC 30, and the display processing unit 40 can be implemented by hardware such as a processor and an electric circuit, and a device such as a memory can be utilized for the implementation as required. The control unit 50 can be implemented by, for example, cooperation of hardware including a CPU, a processor, and a memory, and software (program) for regulating the operation of the CPU and the processor.

The overall structure of the ultrasound diagnostic apparatus of FIG. 1 has been described above. The density-increasing processing in the ultrasound diagnostic apparatus will be now described. In the following description, reference numerals in FIG. 1 will be used when describing the elements (blocks) shown in FIG. 1.

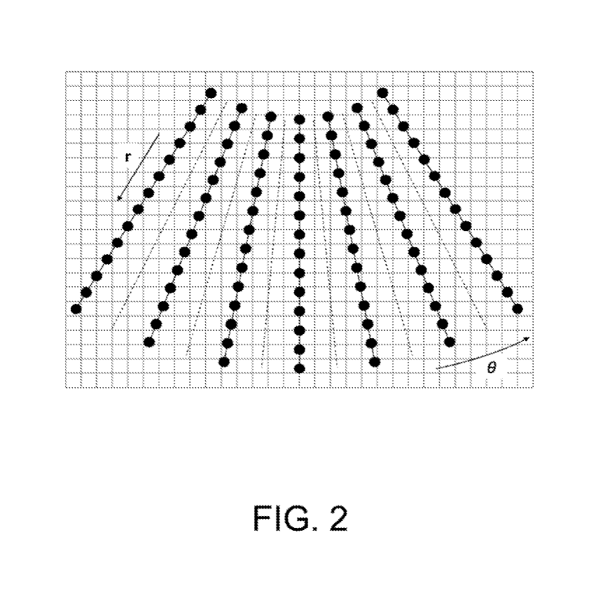

FIG. 2 is a view illustrating specific example imaging data which is obtained by scanning an ultrasound beam. FIG. 2 illustrates imaging data formed of a plurality of line data units corresponding to a plurality of ultrasound beams obtained by scanning an ultrasound beam. FIG. 2 further illustrates a depth direction “r” of the ultrasound beam and the azimuth direction “θ,” which is the scanning direction of the ultrasound beam. A line of a plurality of black circles (solid black circles) arranged in the depth direction r corresponds to the line data.

The line data units are collected along the depth direction “r” of the ultrasound beam. In the depth direction “r,” as the received signals of ultrasound can be sequentially obtained from a shallow portion (on the side near the probe 10) through a deep portion (on the side distant from the probe 10), it is possible to obtain the line data units arranged at a relatively high density. For example, several thousands of line data units can be obtained along a single ultrasound beam. The several thousands of line data units may be used as they are, or several hundreds of line data units obtained by resampling (decimating) the several thousands of line data units may be used.

In the case of convex scanning or sector scanning, for example, the ultrasound beam is scanned in the azimuth direction θ while the angle of the ultrasound beam is shifted stepwise, so that a plurality of ultrasound beams are sequentially formed. In order to obtain one (one frame) two-dimensional B-mode image, for example, approximately several tens to one hundred ultrasound beams are formed, and the line data units are collected for each ultrasound beam along the depth direction “r.”

As described above, while the line data units are collected at a relatively high density along the depth direction “r,” the line data units are separated from each other by a scanning interval of the ultrasound beam in the azimuth direction θ. This makes the density of the imaging data formed of a plurality of line data units relatively low along the azimuth direction θ. Accordingly, the density-increasing processing unit 20, based on the processing which will be detailed below, inserts density-increasing data between adjacent ultrasound beams; that is, on a straight line indicated by a dashed line in FIG. 2, thereby increasing the density of the imaging data.

The density-increasing processing unit 20 places a template corresponding to the azimuth direction θ (the scanning direction of ultrasound beam) within the imaging data, and moves a kernel corresponding to the depth direction “r” of the ultrasound beam to search for a kernel which matches the template, thereby augmenting the density of the scanning direction data which belongs to the template by using the depth direction data which belongs to the kernel which is found.

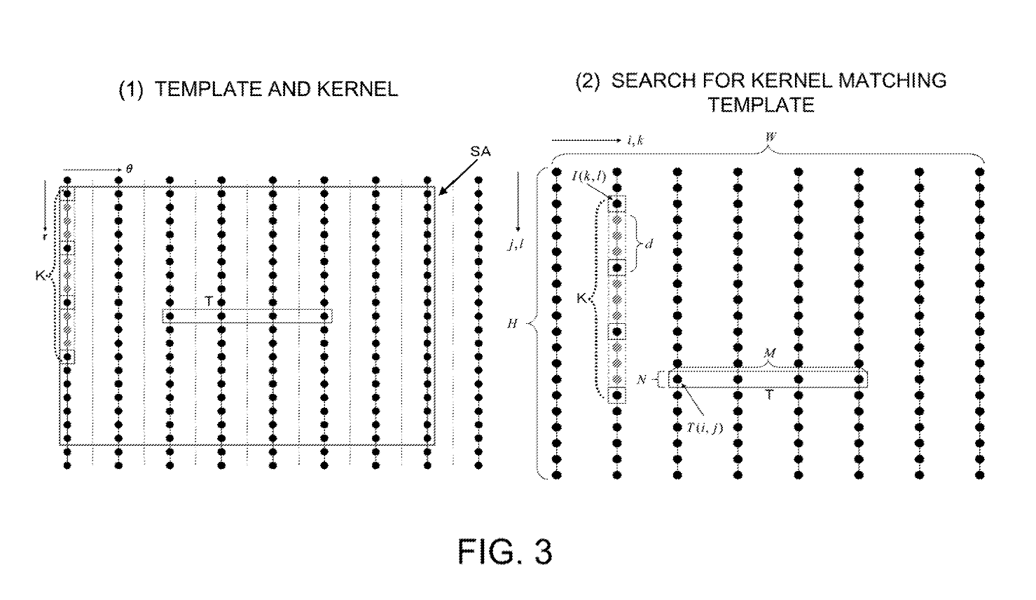

FIG. 3 is a view illustrating specific example search using a template and a kernel. FIG. 3 illustrates the imaging data of FIG. 2. More specifically, FIG. 3 shows the depth direction “r” of the ultrasound beam and the azimuth direction θ which is the scanning direction of the ultrasound beam, and shows a line of a plurality of black circles (solid black circles) arranged in the depth direction “r” as the line data. In FIG. 3, a plurality of line data units obtained in the azimuth direction θ are arranged in parallel to each other.

FIG. 3(1) illustrates a specific example of a template T and a kernel K. In this specific example, the template T has a one-dimensional shape extending in the azimuth direction θ. Provided that data units of the imaging data arranged along the azimuth direction θ are azimuth direction data, the template T includes the azimuth direction data composed of four data units. The template T need not be parallel to the azimuth direction θ so long as the template T has a shape corresponding to the azimuth direction θ, and may be set so as to be inclined with respect to the azimuth direction θ, for example. Also, the shape of the template T is not limited to a one-dimensional shape, and may be a two-dimensional shape (a rectangle or other polygonal shape, or a circular shape). If the imaging data is three-dimensional data, a template T having a three-dimensional shape may be used.

In the specific example illustrated in FIG. 3(1), the kernel K has a one-dimensional shape extending in the depth direction “r.” Provided that data units of the imaging data arranged along the depth direction “r” are depth direction data, the kernel K includes the depth direction data composed of thirteen data units. The kernel K need not be parallel to the depth direction “r” so long as the kernel K has a shape corresponding to the depth direction “r” and may be set so as to be inclined with respect to the depth direction “r,” for example. Also, the shape of the kernel K is not limited to a one-dimensional shape, and may be a two-dimensional shape (a rectangle or other polygonal shape, or a circular shape). If the imaging data is three-dimensional data, a kernel K having a three-dimensional shape may be used. It is desirable that the kernel K and the template T be of identical shape.

The density-increasing processing unit 20 moves the kernel K within the imaging data to search for a kernel K that matches the template. The density-increasing processing unit 20 sets a search area SA within the imaging data, and moves the kernel K within the search area SA which is set. In the specific example of FIG. 3 (1), the search area SA is set as a rectangle enclosing the template T, with the template T being at the center. The shape of the search area SA may, however, be other polygonal shapes or a circular shape. If the imaging data is three-dimensional data, a search area SA having a three-dimensional shape may be adopted. Further, the position of the search area SA is not limited to the example in which the template T is located at the center, and the positional relationship between the template T and the search area SA may be adjusted as appropriate in accordance with the state of the imaging data and other conditions. The size of the search area SA may be fixed or may be adjusted as appropriate in accordance with the state of the imaging data and other conditions. The overall region of the imaging data may be set as the search area SA, for example.

FIG. 3(2) illustrates specific example search for a kernel K that matches a template T. The density-increasing processing unit 20, based on pattern matching between the azimuth direction data that belongs to the template T and the depth direction data that belongs to the kernel K, searches for a kernel K that matches the template T. Specifically, the density-increasing processing unit 20, using pattern matching based on the degree of similarity between the scanning direction data within the template T and the depth direction data selected from within the kernel K at the data intervals of the scanning direction data, searches for the kernel K matching the template T. More specifically, in FIG. 3(2), pattern matching is performed between the template T and the kernel K, with the kernel K being rotated by 90° with respect to the template T. In this case, the kernel K may be rotated by 90° either clockwise or counterclockwise, or the pattern matching may be performed with the kernel K being rotated by 90° both in the clockwise direction and in the counterclockwise direction. In the pattern matching, calculations of the degree of similarity represented by a sum of squared difference of brightness (SSD) shown in Mathematical Formula 1 or a sum of absolute difference of brightness (SAD) shown in Mathematical Formula 2 are used.

$$\begin{matrix}{R}_{SSD} = {\sum }_{q = 0}^{N - 1} {\sum }_{p = 0}^{M - 1} {( I ( k + q , l + d \cdot p ) - T ( i + p , j + q ) )}^{2} \\ \{ \begin{matrix}I ( x , y ) & 0 \leq x \leq W - 1 , & 0 \leq y \leq H - 1 \\ T ( {x}^{′} , {y}^{′} ) & 0 \leq {x}^{′} \leq W - 1 , & 0 \leq {y}^{′} \leq H - 1\end{matrix} & [ Mathematical Formula 1 ] \\ {R}_{SAD} = {\sum }_{j = 0}^{N - 1} {\sum }_{i = 0}^{M - 1} \vert I ( k + q , l + d \cdot p ) - T ( i + p , j + q ) \vert \\ \{ \begin{matrix}I ( x , y ) & 0 \leq x \leq W - 1 , & 0 \leq y \leq H - 1 \\ T ( {x}^{′} , {y}^{′} ) & 0 \leq {x}^{′} \leq W - 1 , & 0 \leq {y}^{′} \leq H - 1\end{matrix} & [ Mathematical Formula 2 ]\end{matrix}$$

Reference signs shown in FIG. 3(2) correspond to variables in Mathematical Formula 1 and Mathematical Formula 2. M and N denote the size of the template T. Specifically, M denotes the size of the template T in the azimuth direction θ; i. e., the number of data units of the azimuth direction data, and N denotes the size of the template T in the depth direction “r”; i.e., the number of lines of the azimuth direction data. In the specific example of FIG. 3(2), M=4 and N=1. T(i, j) denotes a value (pixel value) of each data unit (each pixel) within the template T, where “i” is a coordinate in the azimuth direction θ, and “j” is a coordinate in the depth direction “r.”

I(k, l) denotes a value (pixel value) of each data unit (each pixel) within the kernel K, in which “k” is a coordinate in the azimuth direction θ and “l” is a coordinate in the depth direction “r.” In the kernel K, each data unit of the depth direction data is selected at a data interval of the azimuth direction data within the template T. “d” denotes a data interval in this selection, and in the specific example of FIG. 3(2), d=4, so that every fourth data item is selected within the kernel K along the depth direction “r.”

It is desirable that the template T and the kernel K be of identical size and identical shape within a real space. It is also desirable that the data interval of the azimuth direction data within the template T and the data interval of the depth direction data selected within the kernel K are equal to each other in the real space.

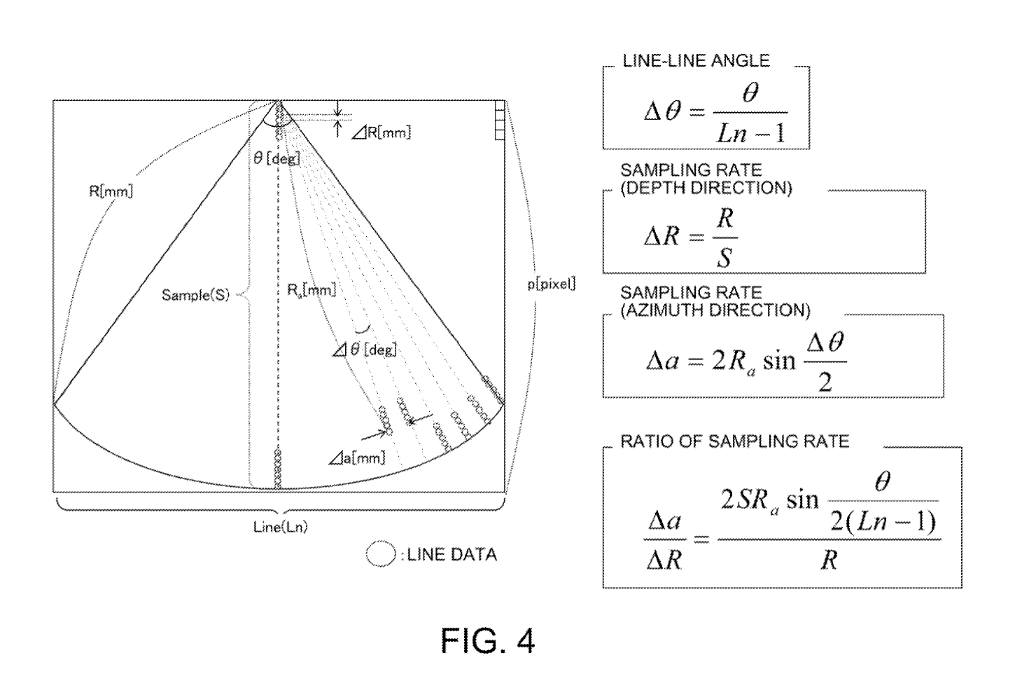

FIG. 4 is a view for explaining the data interval within the real space. FIG. 4 illustrates specific example line data obtained by sector scanning. In the sector scanning and convex scanning, an ultrasound beam is scanned radially or in a sector shape with the probe side being the center, resulting in a larger interval of the ultrasound beam in the deep portion distant from the probe than in the shallow portion close to the probe.

In FIG. 4, the length (maximum depth) of the ultrasound beam is R (millimeters), and the scanning range (angle range) of the ultrasound beam is θ (deg). The number of line data units (the number of samples) which can be obtained along a single ultrasound beam is S, and the number of ultrasound beams (total number of lines) is Ln.

Further, the sampling rate (line data interval) in the depth direction is ΔR. The sampling rate (beam interval) in the azimuth direction depends on the depth, and the sampling rate at the depth Ra is Δa. Accordingly, in order to make the data interval within the template T in the azimuth direction and the interval of data selected from within the kernel K corresponding to the depth direction equal to each other in the real space, the ratio of the sampling rate Δa in the azimuth direction and the sampling rate ΔR in the depth direction indicated in the following formula is used.

$$\frac{Δ a}{Δ R} = \frac{2 {SR}_{a} sin \frac{θ}{2 ( L n - 1 )}}{R} \qquad [ Mathematical Formula 3 ]$$

Assuming that the depth of the template T shown in FIG. 3(2) is Ra, for example, the ratio of the sampling rates is calculated according to Mathematical Formula 3, and the integer which is the closest to the calculation result is set to “d” (a selected interval of the depth direction data) in FIG. 3(2), Mathematical Formula 1, and Mathematical Formula 2. More specifically, the deeper the template T, the greater (wider) the sampling rate Δa in the azimuth direction, and accordingly the greater the selected interval “d” in the depth direction data within the kernel K. Thus, it is possible to make the data interval in the azimuth direction data within the template T and the data interval of the depth direction data selected from within the kernel K equal to each other in the real space.

Referring back to FIG. 3(2), in the pattern matching using the sum of squared difference of brightness (SSD) shown in Mathematical Formula 1, with the kernel K being moved stepwise in the depth direction “r”; e. g., with the kernel K being sequentially moved by an amount corresponding to one data unit arranged at a high density along the depth direction “r,” SSD in Mathematic Formula 1 is calculated between the kernel K and the template T at each position. Further, with the kernel K being shifted along the azimuth direction θ by an amount corresponding to a single ultrasound beam and then moved along the depth direction “r,” SSD in Mathematical Formula 1 is calculated at each position. In this manner, while the kernel K is being moved over the entire region of the search area SA, SSD in Mathematical Formula 1 is calculated. Then, the kernel K at a location within the search area SA where SSD is the minimum value is determined as the kernel K that matches the template T. Here, the kernel K may be moved stepwise at several data intervals along the depth direction “r” and at several beam intervals along the azimuth direction θ.

In the pattern matching using a sum of absolute difference of brightness (SAD) shown in Mathematical Formula 2, similar to the case of the sum of squared difference of brightness (SSD), SAD in Mathematical Formula 2 is calculated at each position while moving the kernel K over the entire region within the search area SA. Then, the kernel K at a location within the search area SA where SAD is the minimum value is determined as the kernel K that matches the template T.

The line data forming the imaging data in FIG. 3(2) has been or has not been subjected to decimation (resampling). If the line data has not been subjected to decimation (before decimation), the large number of depth direction data can result in increased accuracy of the pattern matching, whereas if the line data has been subjected to decimation (after decimation), the thinned-out depth direction data can reduce the operation load of the pattern matching.

Once the kernel K matching the template T has been found, the density-increasing data obtained from the depth direction data of the kernel K is used to increase the density of the azimuth direction data within the template T.

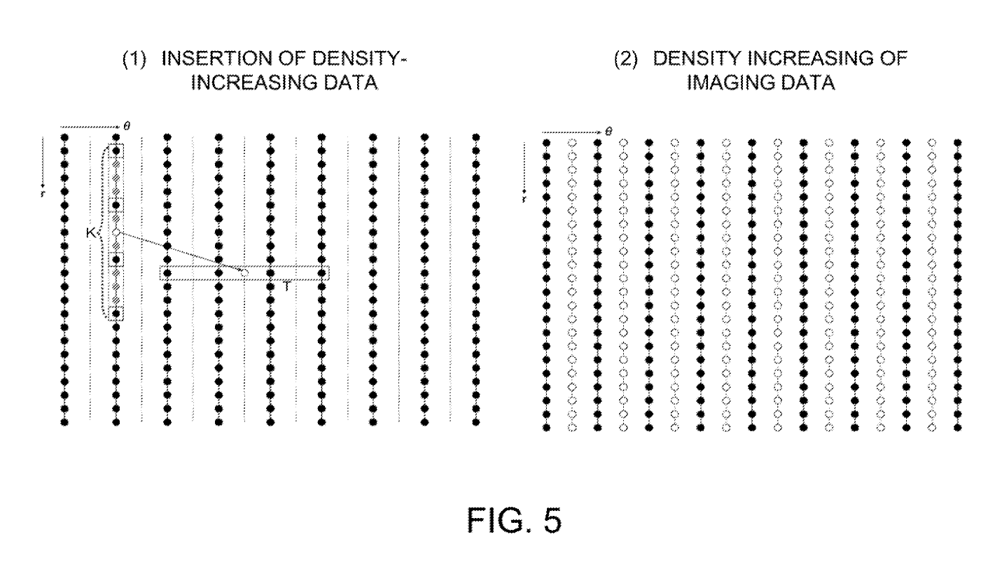

FIG. 5 is a view illustrating a specific example of density increasing by using the density-increasing data. FIG. 5 illustrates the imaging data in FIG. 3. Specifically, FIG. 5 illustrates the depth direction “r” of the ultrasound beam and the azimuth direction θ of the ultrasound beam, and also illustrates the line data as a plurality of black circles (solid black circles) arranged along the depth direction “r.”

FIG. 5(1) illustrates example insertion of the density-increasing data. In FIG. 5(1), a template T and a kernel K matching the template T are shown within the imaging data. The density-increasing processing unit 20 inserts the density-increasing data which can be obtained from the depth direction data within the kernel K matching the template T into a gap of the azimuth direction data within the template T. In the specific example of FIG. 5(1), a depth direction data unit indicated by a blank circle (unfilled circle) located at the center of the kernel K is determined as the density-increasing data unit and is inserted into a gap located at the center of the template T (on the straight line indicated by a dashed line).

The kernel K matching the template T is a kernel K for which the sum of squared difference of brightness (Mathematical Formula 1) or the sum of absolute difference of brightness (Mathematical Formula 2) is the minimum within the search area SA (FIG. 3), and therefore is an image portion which is the most similar to the template T. The template T corresponds to the azimuth direction θ, and the kernel K corresponds to the depth direction “r.” The template T and the kernel K which matches the template T are, although corresponding to different directions, most similar image portions and very likely to exhibit extremely similar properties, including the acoustic behavior of the ultrasound and the nature of a tissue.

Accordingly, as in the specific example illustrated in FIG. 5(1), the density-increasing data unit of a blank circle obtained from the depth direction data of the kernel K which matches the template T is inserted into a gap of the azimuth direction data of the template T. It is desirable that the location of the density-increasing data within the kernel K corresponds to the insertion location of the density-increasing data within the template T. Specifically, as in the specific example illustrated in FIG. 5 (1), for example, it is desirable that the density-increasing data unit obtained from the center of the kernel K is inserted into the center of the template T. Here, the density-increasing data may be selected from the depth direction data units of the kernel K or may be calculated according to an operation based on the depth direction data of the kernel K.

The density-increasing processing unit 20 further places the template T at a plurality of different locations within the imaging data and searches for a kernel K matching the template T at each location, thereby augmenting the density of the azimuth direction data belonging to the template T at the plurality of locations for increasing the density of the imaging data.

FIG. 5(2) illustrates specific example density increasing of the imaging data. In FIG. 5(2), the density-increasing data is inserted into the imaging data over the entire region of the imaging data. Specifically, the specific example of FIG. 5(2) can be obtained by placing the template T at a plurality of locations over the entire region of the imaging data and searching for the kernel K matching the template T at each location to obtain the density-increasing data of a blank circle at each location of the template T and then placing the density-increasing data at each location. In FIG. 5(2), the density-increasing data is inserted so as to fill a gap between adjacent ultrasound beams; i.e. a space on the straight line indicated by a dashed line shown in FIG. 5 (1), thereby increasing the density of the imaging data.

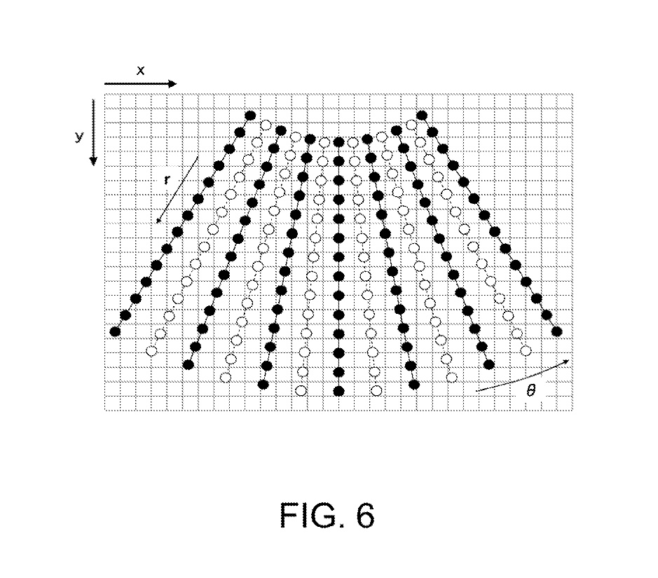

FIG. 6 is a view illustrating a specific example of density-increased imaging data. More specifically, FIG. 6 illustrates imaging data having a density increased by applying to the imaging data illustrated in FIG. 2 the processing which has been described with reference to FIG. 3 to FIG. 5. When compared to the imaging data of FIG. 2, in the imaging data of FIG. 6, the density-increasing data is inserted between adjacent ultrasound beams; i.e., on the straight lines indicated by the dashed lines in FIG. 2, thereby increasing the density of the imaging data. The imaging data having been subjected to density increasing in the density-increasing processing unit 20 is further subjected to coordinate transformation processing in the digital scan converter 30.

The digital scan converter 30, concerning the density-increased imaging data illustrated in FIG. 6, for example, obtains image data corresponding to the display coordinate system of the xy orthogonal coordinates system from the imaging data obtained by the rθ scanning coordinate system corresponding to scanning of the ultrasound beam. In a plurality of coordinates within the xy orthogonal coordinates system shown in a lattice shape in FIG. 6, for example, for each coordinate, interpolation processing using the line data (black circle) and the density-increasing data (blank circle) located near the coordinate is performed to calculate image data in each coordinate of the xy orthogonal coordinates system.

The display processing unit 40 then synthesizes graphic data and other data with respect to the image data thus obtained by the digital scan converter 30 to form a display image, which is displayed on the display unit 42.

While the specific example in which one density-increasing data unit obtained from the center of the kernel K is inserted into the center of the template T has been described with reference to FIG. 5(1), the density-increasing data may be inserted according to a modification example which will be described below.

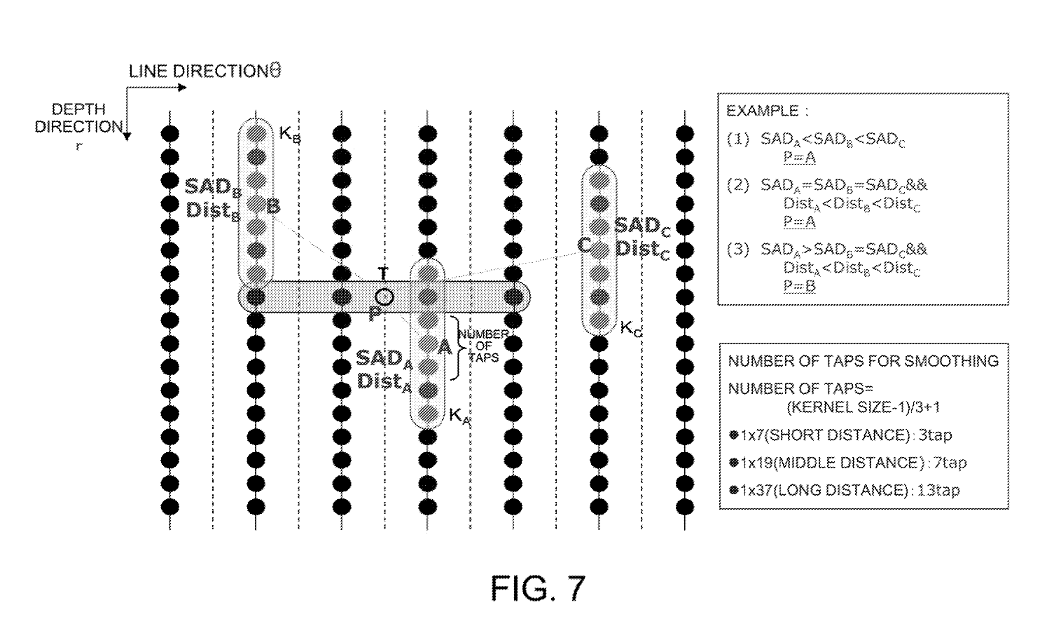

FIG. 7 is a view illustrating example insertion of the density-increasing data in consideration of a distance. FIG. 7 illustrates imaging data to be subjected to density increasing. More specifically, FIG. 7 shows the degree of depth (depth direction) “r” of the ultrasound beam and the line direction (azimuth direction) θ of the ultrasound beam, and also shows a plurality of black circles (solid black circles) arranged along the depth direction “r” as line data units.

In FIG. 7, a template T and a plurality of kernels K_{A}, K_{B}, and K_{C} obtained by search for kernels K corresponding to the template T are shown in the imaging data. FIG. 7 also shows a sum of absolute difference of brightness SAD between the template T and each kernel K, and a distance Dist between the template T and each kernel K (a distance between the centers, for example). Specifically, the sum of absolute difference of brightness and the distance of the kernel K_{A} are SAD_{A} and Dist_{A}, respectively; the sum of absolute difference of brightness and the distance of the kernel K_{B} are SAD_{B} and Dist_{B}, respectively; and the sum of absolute difference of brightness and the distance of kernel K_{C} are SAD_{C} and Dist_{C}, respectively.

In the insertion example of FIG. 7, the density-increasing data P to be inserted into the template T is determined in consideration of the distance Dist in addition to consideration of the SAD indicative of the degree of similarity. Specifically, while the top priority is placed on the SAD being the minimum, if there are a plurality of kernels K with the minimum SAD, the kernel K with the smallest distance Dist is selected. Specific examples will be described below.

(1) If the relationship “SAD_{A}<SAD_{B}<SAD_{C}” is satisfied, the kernel K_{A} is selected, and data A located at the center of the kernel K_{A} is determined as the density-increasing data P which is to be inserted in the template T.

(2) If the relationship “SAD_{A}=SAD_{B}=SAD_{C}” and the relationship “Dist_{A}<Dist_{B}<Dist_{C}” are both satisfied, the kernel K_{A} is selected, and data A located at the center of the kernel K_{A} is determined as the density-increasing data P which is to be inserted in the template T.

(3) If the relationship “SAD_{A}>SAD_{B}=SAD_{C}” and the relationship “Dist_{A}<Dist_{B}<Dist_{C}” are both satisfied, the kernel K_{B} is selected, and data B located at the center of the kernel K_{B} is determined as the density-increasing data P which is to be inserted in the template T.

Further, data obtained by smoothing a plurality of data units from the selected kernel K may be used as the density-increasing data P to be inserted in the template T. If the kernel K_{A} is selected, for example, a mean value of a plurality of data units composed of a data unit A located at the center of kernel K_{A} and data units above and below the data unit A (on the shallow and deep sides thereof) is used as the density-increasing data P. This structure, even if the data unit A is a noise, can reduce or eliminate effects of the noise due to the smoothing, thereby suppressing generation of an unnatural image.

The number of data units (number of taps) for use in smoothing may be determined in accordance with the size of the kernel K. A relationship of “number of taps=(kernel size−1)/3+1”, for example, may be adopted. It is also desirable that the size of the kernel K (the total number of data units in the depth direction within the kernel) is matched with the size of the template T within the real space. In a case where the size of template T in the real space increases as the depth of the template T increases, for example, the size of the kernel K is increased accordingly. As a specific example, when the template T is in a relatively shallow area, the size of the kernel is set to 7, and the number of taps in this case is 3; when the template T is in the middle area, the size of the kernel is set to 19, and the number of taps is 7; and when the template T is in the relatively deep area, the size of the kernel is set to 37, and the number of taps in this case is 13.

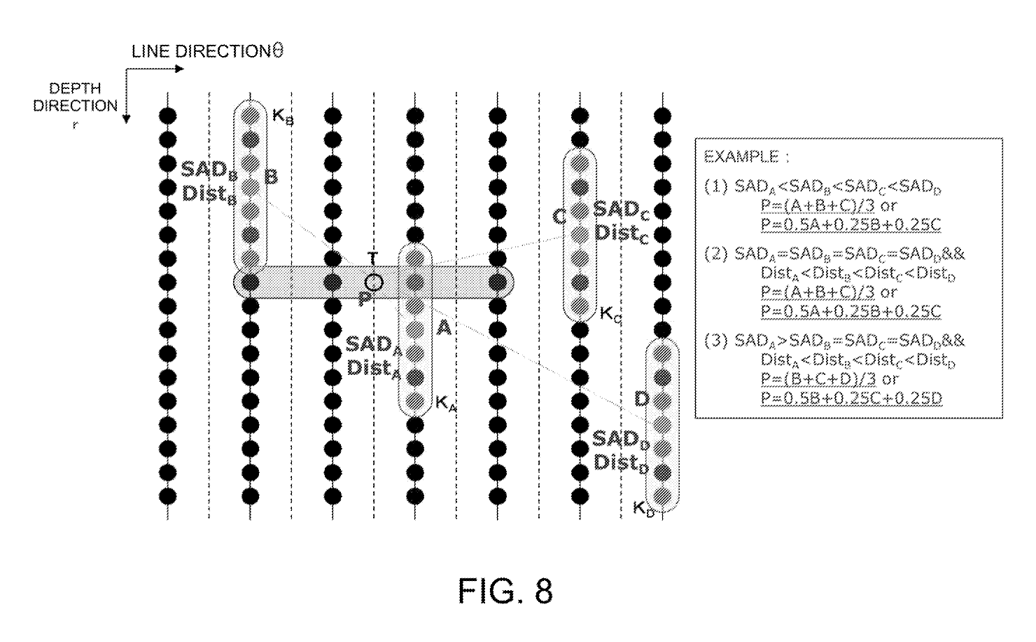

FIG. 8 is a view illustrating example insertion of the density-increasing data using a plurality of kernels K. FIG. 8, similar to FIG. 7, illustrates imaging data to be subjected to density-increasing processing. The imaging data of FIG. 8 includes a template T and a plurality of kernels K_{A}, K_{B}, K_{C}, and K_{D} matching the template T which are obtained in the search for the kernel K.

FIG. 8 shows a sum of absolute difference of brightness SAD between the template T and each kernel K and a distance (center-center distance, for example) Dist between the template T and each kernel K. Specifically, the sum of absolute difference of brightness and the distance of the kernel K_{A} are SAD_{A} and Dist_{A} respectively; the sum of absolute difference of brightness and the distance of kernel K_{B} are SAD_{B} and Dist_{B}, respectively; the sum of absolute difference of brightness and the distance of kernel K_{C} are SAD_{C} and Dist_{C}, respectively; and the sum of absolute difference of brightness and the distance of kernel K_{D} are SAD_{D} and Dist_{D}, respectively.

In the example insertion illustrated in FIG. 8, a plurality of kernels K are selected in consideration of the distance Dist sequentially from a kernel with a smaller SAD which is a degree of similarity. In a case where three kernels K with a smaller SAD are sequentially selected, for example, if a plurality of kernels K have the same SAD value, the kernel with the smallest distance Dist is selected. A specific example will be described below.

(1) In the case of “SAD_{A}<SAD_{B}<SAD_{C}<SAD_{D},” the kernels K_{A}, K_{B}, and K_{C} are selected, and based on the data units A, B, and C located at the centers of the respective kernels K_{A}, K_{B}, and K_{C}, the density-increasing data P to be inserted in the template T is obtained. For example, the mean value of the data units A, B, and C is used as the density-increasing data P. The density-increasing data P may also be obtained by a weighted summation “P=0.5A+0.25B+0.25C” in accordance with the distance of each of the selected kernels K_{A}, K_{B}, and K_{C}.

(2) In the case of “SAD_{A}=SAD_{B}=SAD_{C}=SAD_{D}” and “Dist_{A}<Dist_{B}<Dist_{C}<Dist_{D,}” the kernels K_{A}, K_{B}, and K_{C} are selected, and the density-increasing data P which is to be inserted in the template T is obtained based on the data units A, B, and C located at the centers of the respective kernels K_{A}, K_{B}, and K_{C} A mean value of the data units A, B, and C, for example, is used as the density-increasing data P. The density-increasing data P may also be obtained by a weighted summation “P=0.5A+0.25B+0.25C” in accordance with the distance.

(3) In the case of “SAD_{A}>SAD_{B}=SAD_{C}=SAD_{D}” and “Dist_{A}<Dist_{B}<Dist_{C}<Dist_{D,}” the kernels K_{B}, K_{C}, and K_{D} are selected, and the density-increasing data P which is to be inserted in the template T is obtained based on the data units B, C, and D located at the centers of the respective kernels K_{B}, K_{C}, and K_{D}. A mean value of the data units B, C, and D, for example, is used as the density-increasing data P. The density-increasing data P may also be obtained by a weighted summation “P=0.5B+0.25C+0.25D” in accordance with the distance.

While the specific examples in which the density-increasing data is inserted in the center of the template T have been described, as will be described below, the density-increasing data may be inserted in an insertion location which has been assumed.

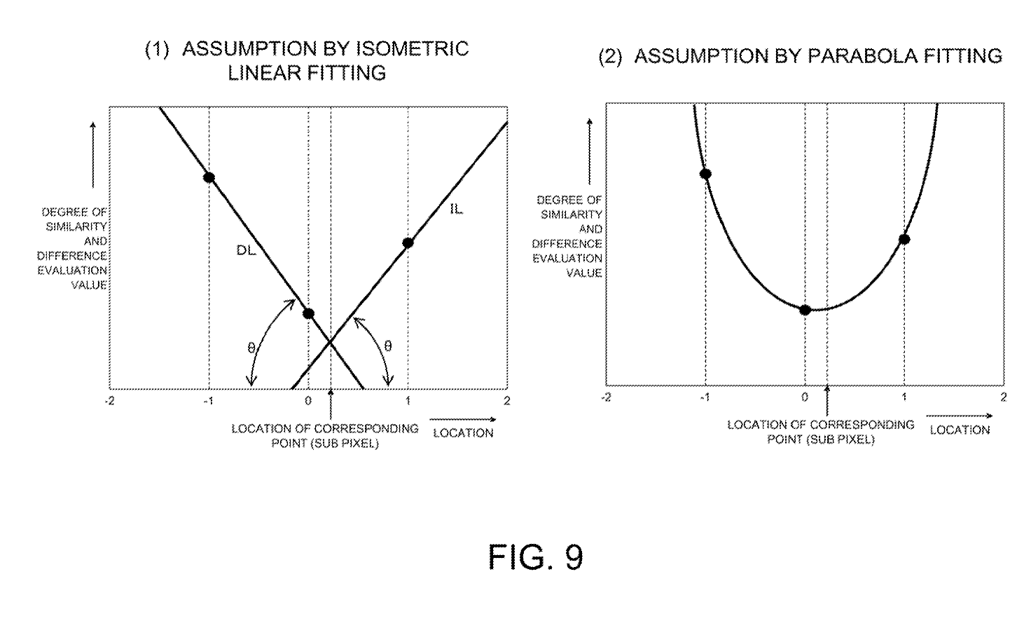

FIG. 9 is a view illustrating a specific example of assumption concerning the insertion location of the density-increasing data. Prior to assumption of the insertion location, the density-increasing processing unit 20 searches for the kernel K matching the template T according to the specific example which has been described above with reference to FIG. 3, for example. In the specific example of assumption illustrated in FIG. 9, the best location in which the density-increasing data is to be inserted is assumed in the gap of the scanning direction data within the template T. The density-increasing processing unit 20 assumes the best location with the best degree of similarity based on a spatial distribution of degree of similarity obtained during the search for the kernel K matching the template T, and inserts the density-increasing data in the assumed best location.

FIG. 9(1) shows an assumption example using isometric linear fitting, and FIG. 9(2) shows an assumption example using parabola fitting. In each of FIGS. 9(1) and (2), the horizontal axis indicates the location of the kernel K, and the vertical axis indicates a value of a degree of similarity at each position, which is value of the sum of squared difference of brightness (Mathematical Formula 1) or a value of the sum of absolute difference of brightness (Mathematical Formula 2), for example. Each black circle (black solid circle) is a specific example of the degree of similarity calculated at each position.

As has been described with reference to FIG. 3, in the search for the kernel K matching the template T, the kernel K at a location where the sum of squared difference of brightness (SSD) or the sum of absolute difference of brightness (SAD) is the minimum value is determined as the kernel K matching the template T.

In FIGS. 9(1) and (2), the location 0 (zero) on the horizontal axis is a search location of the kernel K. More specifically, at the location 0, among a plurality of locations at which the degree of similarity is calculated, the degree of similarity which is calculated is the minimum value. Further, the location 1 and the location −1 on the horizontal axis are shift locations of the kernel K in the vicinity of the location 0 which is the search location. When the degree of similarity is obtained while moving the kernel K by an amount corresponding to one data unit along the depth direction “r,” for example, the shift locations shifted from the location 0 by an amount corresponding to one data unit are the location 1 and the location −1.

The density-increasing processing unit 20, based on the spatial distribution of the degree of similarity in the vicinity of the search location, assumes a corresponding point location (best position) at which the degree of similarity is the best. As in the example illustrated in FIG. 9(1), for example, the isometric fitting is used to assume the corresponding point location. More specifically, a decreasing straight line DL showing a decrease in the degree of similarity from the negative direction side toward the position direction side and an increasing straight line IL showing an increase in the degree of similarity from the negative direction side toward the position direction side are set such that, with the inclination θ of the decreasing straight line DL and the inclination θ of the increasing straight line IL being the same (isometric), the decreasing straight line DL and the increasing straight line IL pass the three points (black circle) at the positions −1, 0, and 1, and the position of the intersection of the decreasing straight line DL and the increasing straight line IL thus set is determined as the corresponding point location (sub pixel position).

Parabola fitting may also be used as in the example illustrated in FIG. 9(2), for example. More specifically, a parabola, for example, passing through three points (black circles) at the locations −1, 0, and 1 is set, and a location at which the parabola is a relative minimum is determined as the corresponding point location (sub pixel location).

As described above, the corresponding point location with a more preferable degree of similarity (smaller SSD or SAD) than that of the location 0 which is the search location is assumed. Upon assumption of the corresponding point location, the density-increasing processing unit 20 inserts the density-increasing data obtained from the kernel K of the search location into the corresponding point location within the template T. For example, the density-increasing data obtained from the center of the kernel K is inserted in a location which is shifted from the center of the template T by a distance corresponding to the corresponding point location.

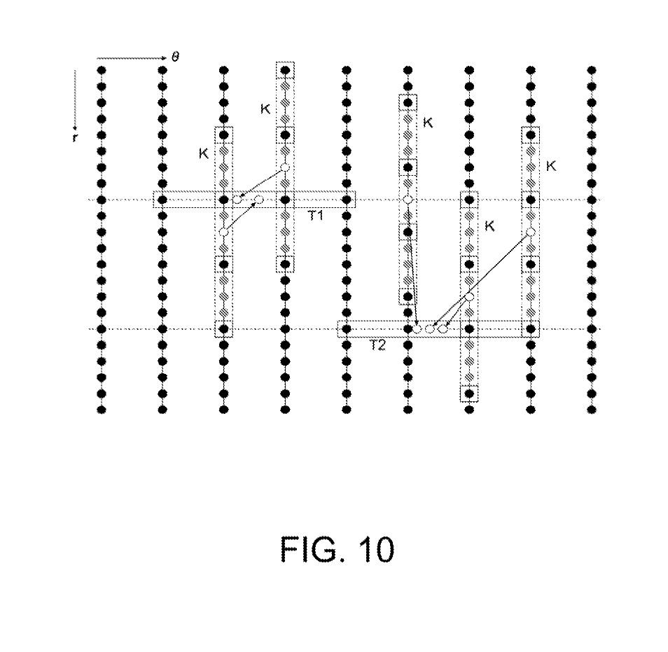

FIG. 10 is a view illustrating example insertion of the density-increasing data into the corresponding point location. FIG. 10 illustrates imaging data to be subjected to density increasing. Specifically, FIG. 10 shows the depth direction “r” of the ultrasound beam and the azimuth direction θ which is the scanning direction of the ultrasound beam, and a plurality of black circles (solid black circles) arranged along the depth direction “r” as the line data units.

The imaging data illustrated in FIG. 10 includes two templates T1 and T2 and kernels K matching these templates. In the template T1, two density-increasing data units (blank circles) obtained from two kernels K are inserted in a gap in the azimuth direction data (between the scanning lines). Further, in the template T2, three density-increasing data units obtained from three kernels K are inserted in a gap of the azimuth direction data. The insertion location of each density-increasing data unit is assumed based on the processing which has been described with reference to FIG. 9. As illustrated in FIG. 10, a plurality of density-increasing data units may be inserted between data units in a single template T.

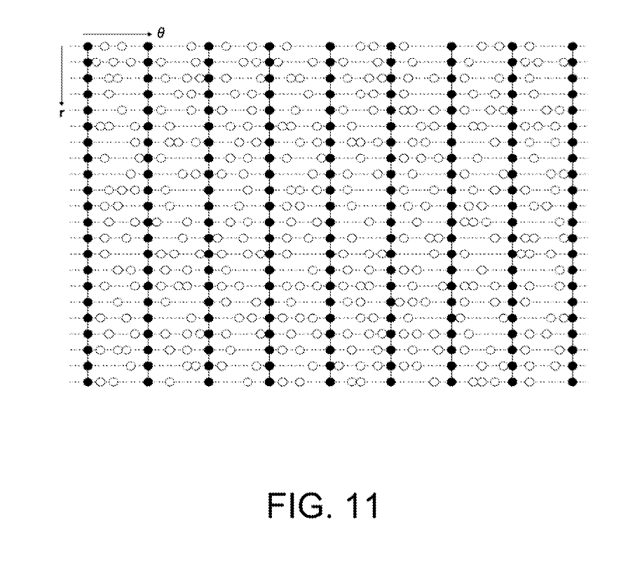

FIG. 11 is a view illustrating specific example density increasing using the corresponding point locations. In FIG. 11, the density-increasing data units are inserted in an overall region of the imaging data. In other words, the specific example of FIG. 11 can be obtained by placing templates T at a plurality of positions over the entire region of the imaging data, searching for kernels K matching the template T at each position to obtain the density-increasing data unit of a blank circle from the kernel K, and placing the density-increasing data unit in the corresponding point location. In FIG. 11, a plurality of density-increasing data units are inserted between adjacent ultrasound beams; that is, between the line data units indicated by black circles in FIG. 11, thereby increasing the density of the imaging data.

The density-increasing data units may be inserted at a uniform density within the imaging data or at various densities in accordance with the depth. In the imaging data obtained by sector scanning or convex scanning, for example, as the interval of the ultrasound beams are increased at a deeper portion, the number of density-increasing data units may be increased at a deep portion while the density increasing processing is omitted in a shallow portion.

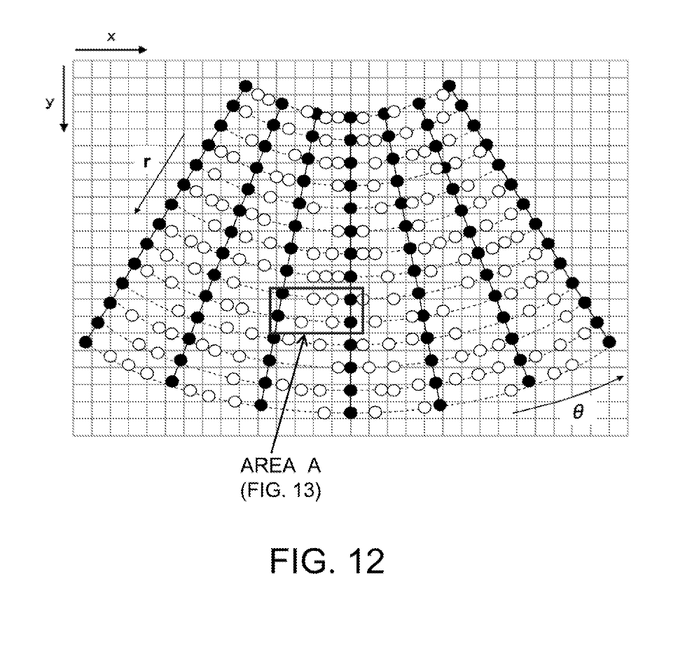

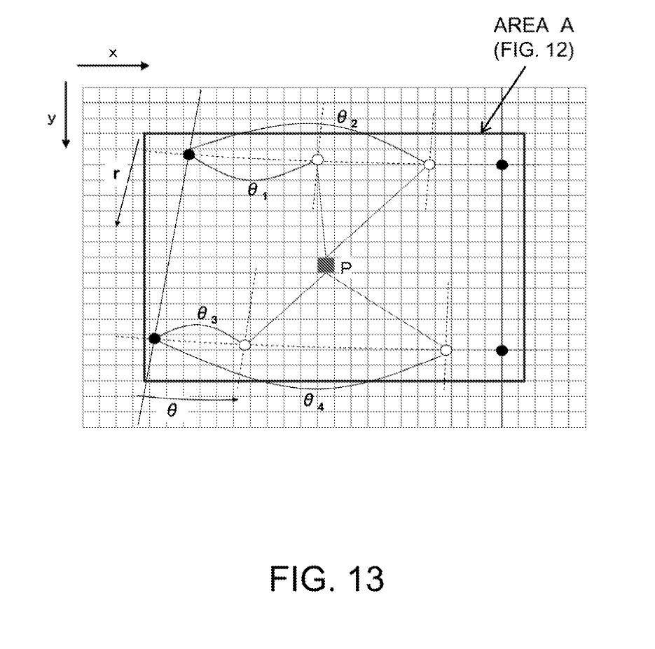

FIG. 12 is a view illustrating imaging data having been subjected to density increasing using the corresponding point locations. More specifically, FIG. 12 illustrates density-increased imaging data obtained by applying to the imaging data illustrated in FIG. 2 the processing which has been described with reference to FIG. 9 to FIG. 11. When compared to the imaging data of FIG. 2, in FIG. 12, a plurality of density-increasing data units are inserted between adjacent ultrasound beams; that is, between the line data units indicated by black circles, to thereby increase the density of the imaging data several-fold. The imaging data having been subjected to density increasing by the density-increasing processing unit 20 is further subjected to coordinate transformation processing in the digital scan converter 30.

The digital scan converter 30, concerning the density-increased imaging data illustrated in FIG. 12, for example, obtains image data corresponding to the display coordinate system of the xy orthogonal coordinate system from the imaging data obtained with the rθ scanning coordinate system corresponding to the ultrasound beam. In a plurality of coordinates in the xy orthogonal coordinates system shown in a lattice shape in FIG. 12, for example, for each coordinate, interpolation processing using the line data (black circle) and the density-increasing data (blank circle) in the vicinity of that coordinate is performed to calculate image data in each coordinate of the xy orthogonal coordinate system.

FIG. 13 is a view illustrating specific example interpolation processing performed in the digital scan converter (DSC) 30. FIG. 13 illustrates an enlarged view of the area A in FIG. 12. In order to obtain pixel data P forming the image data of the xy orthogonal coordinate system, the digital scan converter 30 uses at least one of the line data (black circle) and the density-increasing data (blank circle) located in the vicinity of the pixel data P.

In the specific example illustrated in FIG. 13, four density-increasing data units which are selected as four data units that are the closest to the pixel data P are used. The location of the each density-increasing data unit (corresponding point location) has been assumed according to the processing described with reference to FIG. 9 and stored in a memory, for example. The digital scan converter 30 reads the corresponding point locations (θᵣ, θ₂, θ₃, θ₄) of the four density-increasing data units from the memory, and obtains the pixel data P from the four density-increasing data units, based on weighted summation in accordance with the distance from the location of the pixel data P to each density-increasing data unit, for example. While in the specific example of FIG. 13, four density-increasing data units are used to obtain the pixel data P, the four data units used for the interpolation processing may include the line data, depending on the location of the pixel data P.

FIG. 14 is a flowchart showing a summary of the processing performed by the ultrasound diagnostic apparatus of FIG. 1. When imaging data formed of a plurality of line data units corresponding to a plurality of ultrasound beams are obtained (S1401), the density-increasing processing unit 20 places a template T within the imaging data (S1402, FIG. 3), and sets a search area SA (S1403, FIG. 3). The density-increasing processing unit 20 also sets a data interval of the depth direction data to be selected in a kernel K, in accordance with the location (depth) of the template T (S1404, FIG. 4).

The density-increasing processing unit 20 moves the kernel K within the search area SA (S1405, FIG. 3), and, while moving the kernel, conducts pattern matching between the kernel K and the template T at each location of the kernel K (S1406, FIG. 3). When the pattern matching is completed for the overall region of the search area SA and a kernel K matching the template T is found (S1407), density-increasing data obtained from the depth direction data of the matching kernel K is inserted in a gap in the azimuth direction data within the template T (S1408, FIG. 5, and FIG. 7 to FIG. 11).

The density-increasing processing unit 20 places the template T at a plurality of locations within the imaging data, and executes the processing in steps S1402 to S1408 at each location. The processing in steps S1402 to S1408 is executed repeatedly until the processing for all the templates in the overall region of the imaging data is completed (S1409).

When the density-increasing data is inserted in the overall region within the imaging data in this manner by the density-increasing processing unit 20, the density-increased imaging data is converted to the display coordinate system by the digital scan converter 30 (S1410, FIGS. 6, 12, and 13), and a density-increased image is displayed on the display unit 42 (S1411).

The ultrasound diagnostic apparatus illustrated in FIG. 1 augments the density of the scanning direction data (azimuth direction data) arranged at a low density along the scanning direction (azimuth direction) of the ultrasound beam, based on the depth direction data arranged at a high density along the depth direction of the ultrasound beam, thereby increasing the density of the imaging data. It is therefore possible to provide an ultrasound image having a relatively high resolution. It is also possible to increase the density of a moving image obtained at a high frame rate and low density, for example, to thereby provide a moving image with a high frame rate and high density. Further, in addition to density increasing at a deep portion of an image obtained by sector scanning or convex scanning, the density of an image obtained by linear scanning and so on may also be increased.

A part or all of the functions of the density-increasing processing unit 20 through the display processing unit 40 illustrated in FIG. 1 may be implemented by a computer according to a program corresponding to a part or all of the processing which has been described with respect to FIG. 3 to FIG. 14, to thereby cause the computer to function as an ultrasound image processing apparatus. The above-described program is stored in a computer-readable storage medium such as a disk or a memory, for example, and is provided to the computer through the storage medium. Of course, such a program may be provided to the computer through an electrical communication line such as the Internet.

The ultrasound diagnostic apparatus illustrated in FIG. 1, which is a preferable embodiment of the present invention, has been described in detail. A specific example of an ultrasound image obtained by the ultrasound diagnostic apparatus of FIG. 1 will be described below.

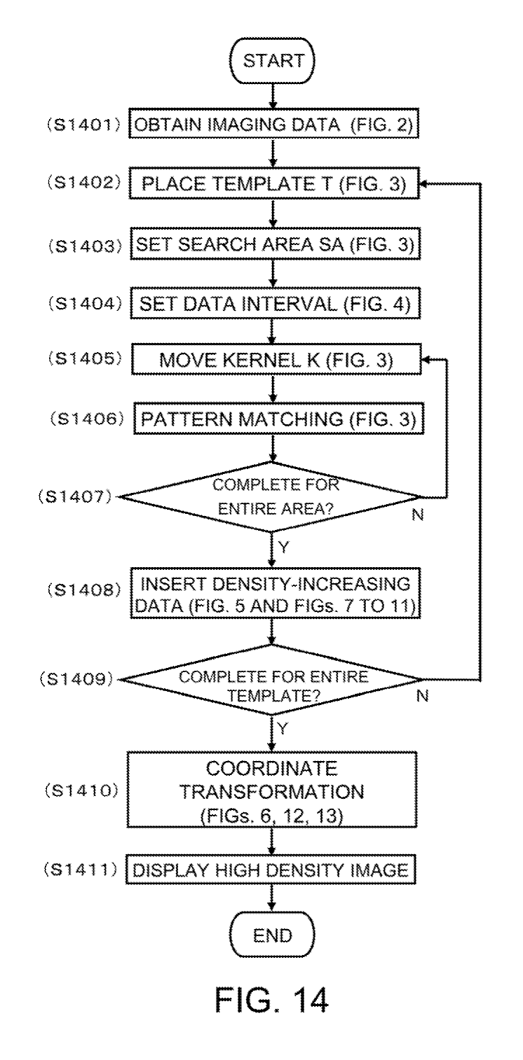

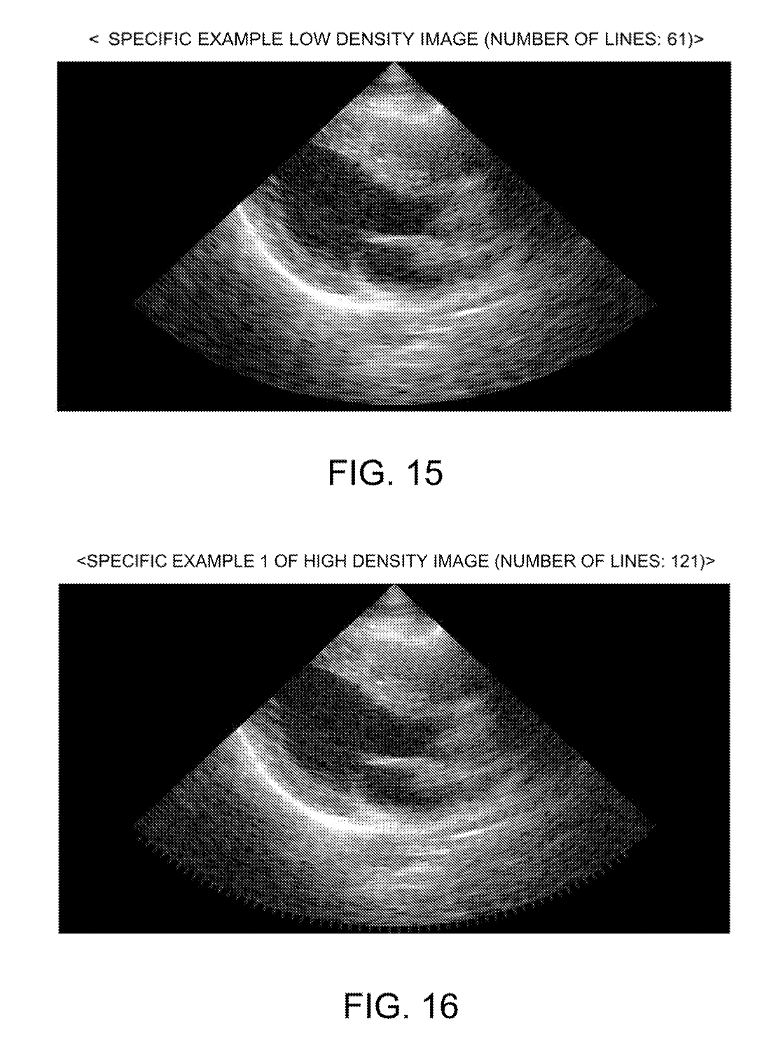

FIG. 15 is a view illustrating a specific example low-density image. The low-density image of FIG. 15 is a B-mode image whose number of lines (the number of beams), is 61, obtained by sector scanning. Specific example high-density images obtained by increasing the density of the low-density image of FIG. 15 are shown in FIG. 16 to FIG. 19.

FIG. 16 is a view illustrating a specific example 1 of a high-density image. The high-density image of FIG. 16 is a high-density image whose number of lines is 121, obtained by sequentially inserting a single density-increasing data unit obtained from a single kernel K with the minimum SAD value into the low-density image of FIG. 15, according to the example insertion of density-increasing data which has been described with reference to FIG. 7.

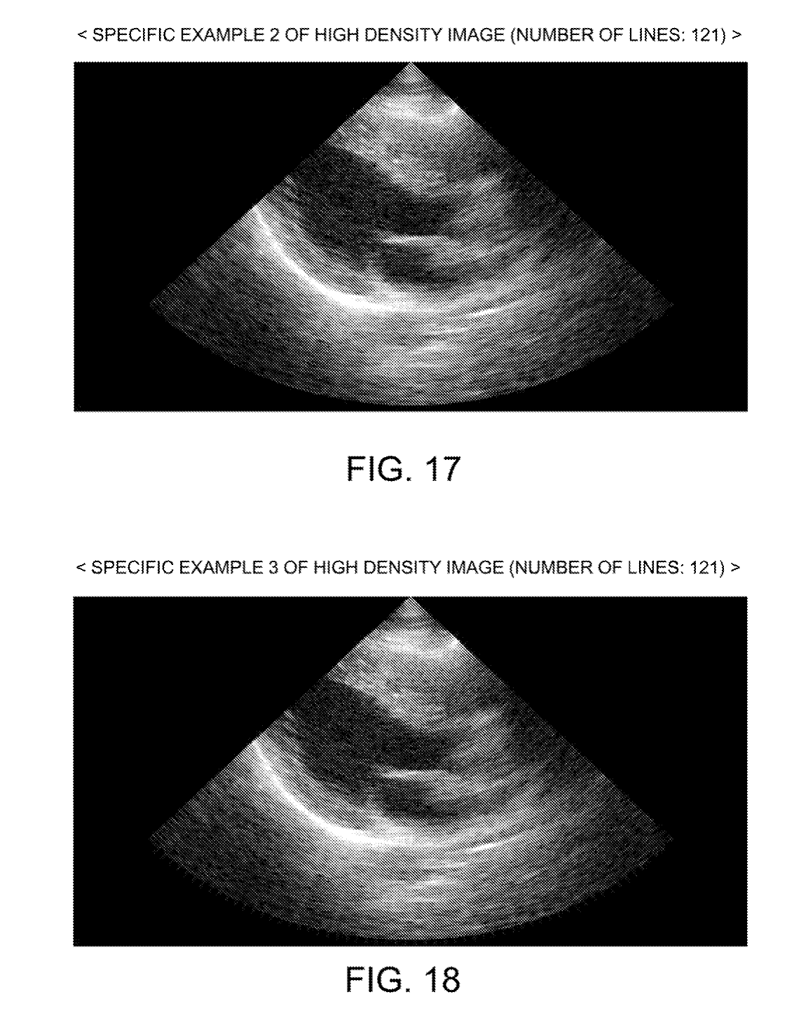

FIG. 17 is a view illustrating a specific example 2 of a high-density image. The high-density image of FIG. 17 is a high-density image obtained by sequentially inserting a density-increasing data unit obtained by smoothing data from a single kernel K with the minimum SAD value into the low-density image of FIG. 15, according to the example insertion of density-increasing data which has been described with reference to FIG. 7.

FIG. 18 is a view illustrating a specific example 3 of a high-density image. The high-density image of FIG. 18 is a high-density image obtained by sequentially inserting a density-increasing data unit obtained from an average value of data units from three kernels K with small SAD values into the low-density image of FIG. 15, according to the example insertion of density-increasing data which has been described with reference to FIG. 8.

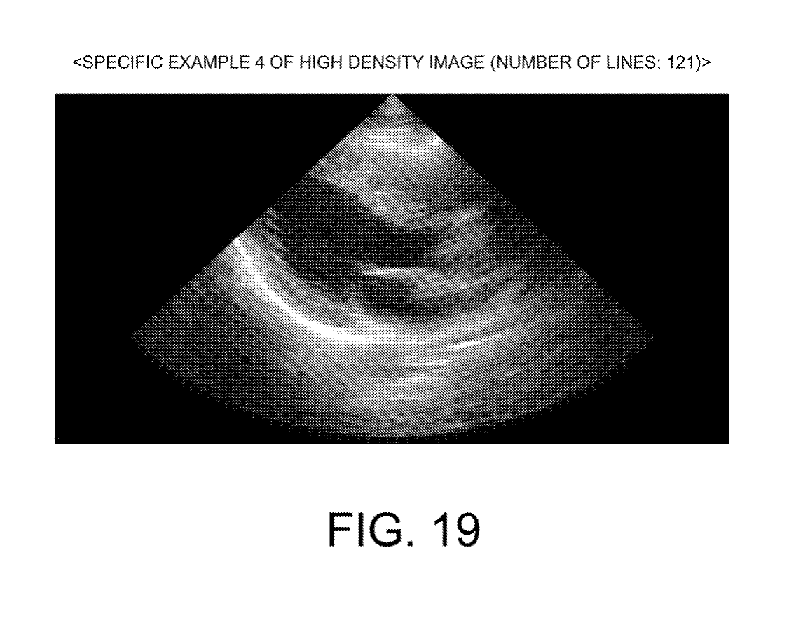

FIG. 19 is a view illustrating a specific example 4 of a high-density image. The high-density image of FIG. 19 is a high-density image obtained by sequentially inserting a density-increasing data unit obtained by applying weighted summation in accordance with a distance to the data units from three kernels K with small SAD values into the low-density image of FIG. 15, according to the example insertion of density-increasing data which has been described with reference to FIG. 8.

All of the high-density images illustrated in FIG. 16 to FIG. 19 have a higher resolution and are clearer than the low-density image of FIG. 15.

Specific examples of ultrasound image which can be obtained from the ultrasound diagnostic apparatus of FIG. 1 have been described above. The ultrasound diagnostic apparatus (the present ultrasound diagnostic apparatus) illustrated in FIG. 1 further has additional or modified functions, which will be described below.

### Filter Processing in Depth Direction

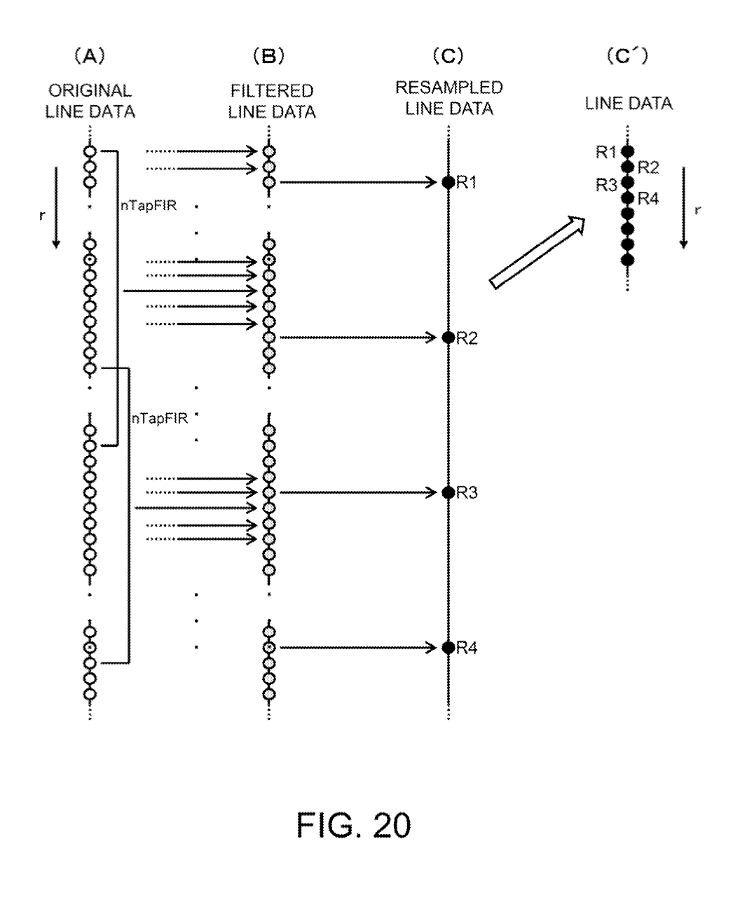

FIG. 20 is a view for explaining various processing with respect to the line data. The various processing illustrated in FIG. 20 is executed, for example, by the transmitter/receiver unit 12 or the density-increasing processing unit 20.

FIG. 20(A) illustrates the original line data obtained by the transmitter/receiver unit 12. The original line data illustrated in FIG. 20 (A) is data corresponding to one ultrasound beam (received beam), and is composed of approximately several hundred to several thousand sampling data units.

The present ultrasound diagnostic apparatus applies filter processing in the depth direction “r” to the original line data. FIR filter processing with respect to some sampling data units arranged in the depth direction “r,” for example, is applied. FIG. 20(A) illustrates, as specific example filter processing, an n-Tap (tap) FIR filter with respect to n (n is a natural number) sampling data units. The line data having been filtered as illustrated in FIG. 20(B) can be obtained by shifting the window of n-TapFIRfilter (range of n data units) by one data unit in the depth direction “r” to thereby sequentially obtain the filtered data, for example.

The present ultrasound diagnostic apparatus applies resampling processing to the filtered line data illustrated in FIG. 20(B), to obtain resampled line data illustrated in FIG. 20(C). For example, sampling data units are extracted at intervals of several data units from the filtered line data arranged in the depth direction “r.”

The resampled line data illustrated in FIG. 20(C) may be obtained directly from the original line data illustrated in FIG. 20(A) by shifting the n-TapFIRfilter by several data units to obtain the filtered data.

The present ultrasound diagnostic apparatus uses the resampled line data illustrated in FIG. 20(C); that is, the line data illustrated in FIG. 20(C′), to perform the density increasing processing of the imaging data. The imaging data having the density thereof increased by the processing described with reference to FIG. 3 to FIG. 13 is obtained, for example. Further, the present ultrasound diagnostic apparatus applies filter processing in the depth direction “r” to the density-increased imaging data.

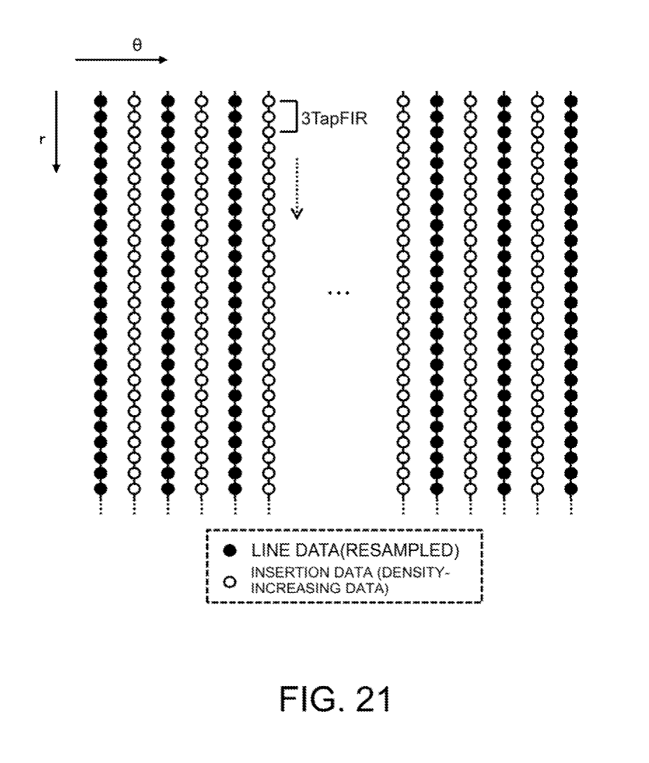

FIG. 21 is a view for explaining the filtering processing in the depth direction “r” with respect to the density-increased imaging data. FIG. 21 illustrates density-increased imaging data. More specifically, FIG. 21 illustrates the depth direction “r” of the ultrasound beam and the azimuth direction θ of the ultrasound beam, and a plurality of black circles (solid black circles) arranged in the depth direction “r” are resampled line data units (see FIG. 20(C′)), and a plurality of blank circles (unfilled circles) arranged in the depth direction “r” are data units (density-increasing data units) inserted by the density-increasing processing (see FIG. 3 to FIG. 13, for example).

In the ultrasound diagnostic apparatus, the density-increasing processing unit 20, for example, applies the filtering processing to the density-increasing data (blank circle) which is substantially the same as the filtering processing in the depth direction “r” applied to the line data (black circles). The term “substantially the same” as used herein refers to cases where the lengths of a filter (the number of data units) within the real space are the same or substantially the same, and weights with respect to each data unit (filter coefficients) are the same or substantially the same, for example.

Specifically, in a case where the n-TapFIRfilter illustrated in FIG. 20(A) is used with respect to the line data, a 3-Tap(tap)FIRfilter for 3 target data units is applied to the density-increasing data, as illustrated in FIG. 21. The filter length of the n-TapFIRfilter illustrated in FIG. 20(A) is n data units, and the length thereof within the real space corresponds to three data units (R1 to R3, for example) in FIG. 20(C). Therefore, a 3-TapFIRfilter having a length corresponding to three line data units (black circles) is applied to the density-increasing data units (blank circles) illustrated in FIG. 21.

Further, the coefficient of the top data, the coefficient of the center data, and the coefficient of the last data of the n-TapFIRfilter (FIG. 20) are subjected to standardization processing, and are used as the coefficient of the top data and the coefficient of the center data of the 3-TapFIRfilter (FIG. 21).

It should be noted that the filter length or weight described above is only one specific example, and the filter length or weight is not limited to that specific example. Also, the user may adjust the filter length or weight.

### Pattern Matching in Consideration of Brightness Bias

In the description with reference to FIG. 3 described above, concerning the search for a kernel K matching a template T, pattern matching using the sum of squared difference of brightness (SSD) indicated by Mathematical Formula1 or the sum of absolute difference of brightness (SAD) indicated by Mathematical Formula 2 has been described.

The present ultrasound diagnostic apparatus is capable of locally adjusting the gain within an ultrasound image based on gain adjustment in accordance with the depth (STC, for example) or gain adjustment in the azimuth direction (ANGLEGAIN, for example) within the apparatus. It is therefore desirable to use an evaluation value which is robust concerning brightness (degree of brightness) in the pattern matching. Accordingly, the present ultrasound diagnostic apparatus may define ZSAD (Zero-mean Sum of Absolute Difference) in the following formula, and use the ZSAD in the following formula in the pattern matching.

$${R}_{ZSAD} = {\sum }_{q = 0}^{N - 1} {\sum }_{p = 0}^{M - 1} \vert ( T ( i + p , j + q ) - \overline{T} ) - ( I ( k + q , l + d \cdot p ) - \overline{I} ) \vert \\ \overline{T} = \frac{1}{NM} {\sum }_{q = 0}^{N - 1} {\sum }_{p = 0}^{M - 1} T ( i + p , j + q ) \\ \overline{I} = \frac{1}{NM} {\sum }_{q = 0}^{N - 1} {\sum }_{p = 0}^{M - 1} I ( k + q , l + d \cdot p ) \\ \{ \begin{matrix}T ( x , y ) & 0 \leq x \leq W - 1 , & 0 \leq y \leq H - 1 \\ I ( {x}^{′} , {y}^{′} ) & 0 \leq {x}^{′} \leq W - 1 , & 0 \leq {y}^{′} \leq H - 1\end{matrix} \qquad [ Mathematical Formula 4 ]$$

Reference signs shown in FIG. 3(2) corresponding to variables in Mathematical Formula 4. M and N, for example, represent the size of a template T. Specifically, M represents a size of the template T in the azimuth direction θ; that is, the number of azimuth direction data units, and N represents the size of the template T in the depth direction “r”; that is, the number of lines of the azimuth direction data units. In the specific example of FIGS. 3(2), M=4, and N=1. T(i, j) represents a value (pixel value) of each data unit (each pixel) within the template T, in which “i” represents a coordinate in the azimuth direction θ, and “j” represents a coordinate in the depth direction “r.”

Further, I(k, l) represents a value (pixel value) of each data unit (each pixel) of a kernel K, in which “k” represents a coordinate in the azimuth direction θ, and “1” represents a coordinate in the depth direction “r.” Within the kernel K, each data unit of the depth direction data is selected at a data interval of the azimuth direction data in the template T. “d” represents the data interval for such a selection, and, in the specific example illustrated in FIG. 3(2), d=4, so that every fourth data item is selected within the kernel K along the depth direction “r.”

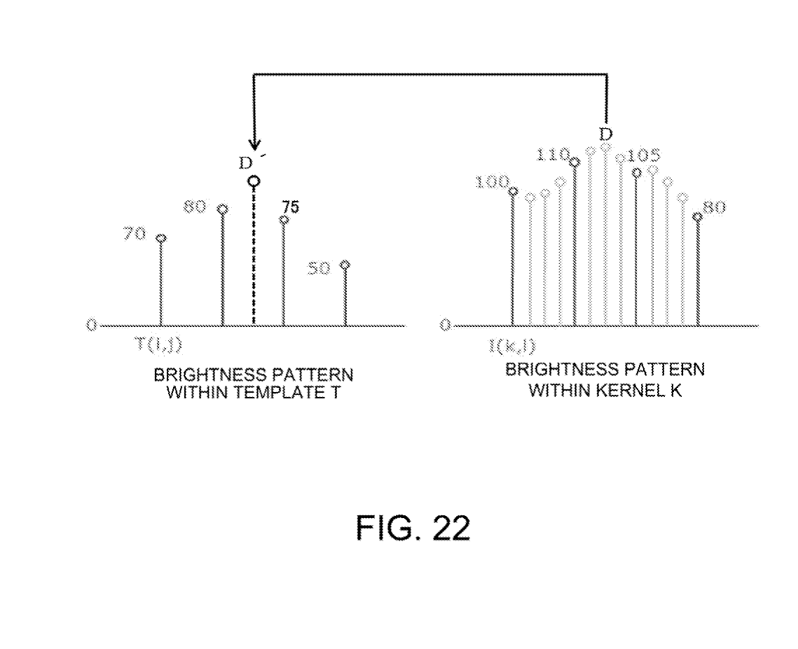

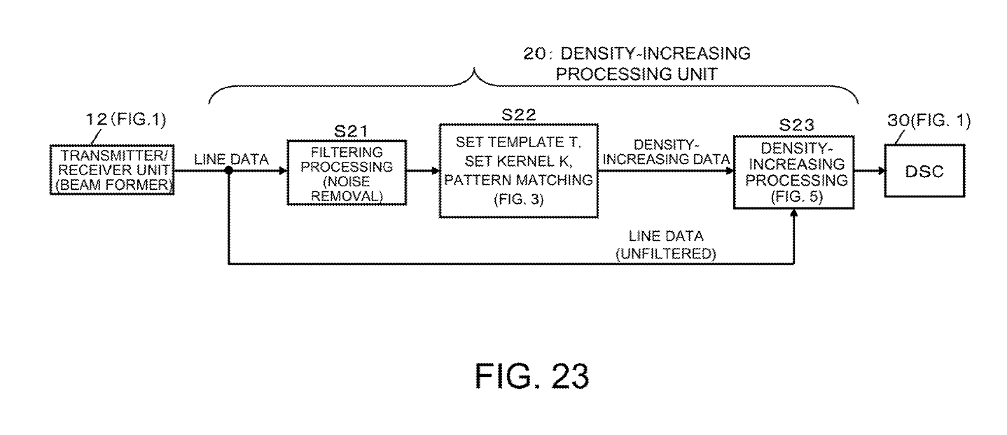

FIG. 22 is a view illustrating a specific example of pattern matching. FIG. 22 illustrates a specific example of a brightness pattern (pixel values 70, 80, 75, 50) within the template T and a brightness pattern (pixel values 100, 110, 105, 80) within the kernel K.

Using the SAD of Mathematical Formula 2 in the specific example illustrated in FIG. 22 results in R_{SAD}=120. On other hand, using the ZSAD of Mathematical Formula 4 in the specific example illustrated in FIG. 22 results in R_{ZSAD}=0, which increases the possibility that the kernel K in FIG. 22 is selected as a kernel K matching the template T in FIG. 22.

In a case where, in the specific example illustrated in FIG. 22, a pixel D (pixel value D) within the kernel K is inserted between pixels within the template T and used as a pixel D′ (pixel value D), the pixel value will be determined based on the following formula.

$${D}^{′} = D - \overline{I} + \overline{T} \\ \overline{I } … \overline{I} = \frac{1}{NM} {\sum }_{q = 0}^{N - 1} {\sum }_{p = 0}^{M - 1} I ( k + q , l + d \cdot p ) in Formula 4 \\ \overline{T } … \overline{T} = \frac{1}{NM} {\sum }_{q = 0}^{N - 1} {\sum }_{p = 0}^{M - 1} T ( i + p , j + q ) in Formula 4 \qquad [ Mathematical Formula 5 ]$$

### Pattern Matching Based on Filtered Data

FIG. 23 is a view for explaining a modification example of the processing performed in the density-increasing processing unit 20. In the modification example illustrated in FIG. 23, the density-increasing processing unit 20 applies filtering processing intended for noise removal or smoothing to the line data obtained from the transmitter/receiver unit 12 illustrated in FIG. 1 (S21). With this processing, noises which adversely affect the pattern matching are removed.

The density-increasing processing unit 20 subsequently sets a template T and a kernel K and executes pattern matching processing within the imaging data based on the line data from which noise has been removed (see S22, and FIG. 3). As a result, the density-increasing data units to be inserted between the line data units are selected.

The density-increasing processing unit 20 then inserts the line data from the transmitter/receiver unit 12 corresponding to the locations selected in step S22 into the imaging data based on the line data obtained from the transmitter/receiver unit 12, as the density-increasing data, thereby increasing the density of the imaging data (see S23, and FIG. 5). The density-increased imaging data is then output to the digital scan converter (DSC) 30 illustrated in FIG. 1.

In the modification example illustrated in FIG. 23, in which pattern matching is performed based on the line data having been filtered in S21, a reduction in the accuracy of pattern matching caused by noise can be suppressed.

### Extension of Search Area SA

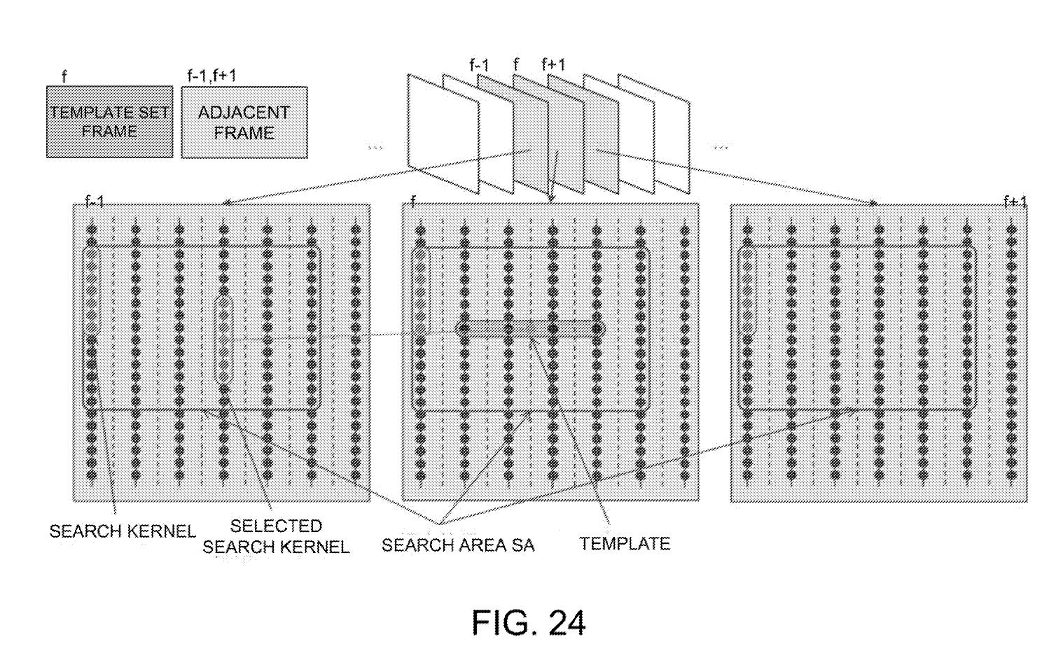

FIG. 24 is a view for explaining a modification example in which the search area SA is extended. FIG. 24 illustrates the imaging data obtained based on the line data in a plurality of frames. In FIG. 24, a frame “f” is a frame of interest which is a subject of density-increasing processing, and a template is set within the imaging data of the frame “f.”

In the modification example illustrated in FIG. 24, a kernel matching the template of the frame “f” is searched for in frames other than the frame “f,” in addition to the frame “f.” For example, a search area SA is set within the frame “f,” and search areas SA are further set within a frame “f−1” and a frame “f+1” adjacent to the frame “f,” and the kernels matching the template of frame “f” are searched for within the search areas SA set in the frame “f,” the frame “f−1,” and the frame “f+1.”

This structure increases the accuracy of pattern matching when compared to the structure in which a kernel is searched for only within a frame in which a template is set. Here, frames used for the search are not limited to those adjacent to the frame of interest in which a template is set, and may be extended to a range which is distant from the frame of interest by several frames, for example.

In calculations of the degree of similarity (Mathematical Formulas 1, 2, and 4), the frame of interest and other frames may be weighted in different manners. For example, a kernel matching a template may be searched for, with the maximum weight being applied to the frame of interest and a smaller weight being applied to a frame further distant from the frame of interest.

### REFERENCE SIGN LIST

10 probe, 12 transmitter/receiver unit, 20 density-increasing processing unit, 30 digital scan converter (DSC), 40 display processing unit, 42 display unit, 50 control unit.

1. An ultrasound diagnostic apparatus, comprising:
a probe configured to transmit and receive an ultrasound;
a transmitter/receiver unit configured to control the probe to scan an ultrasound beam;
a density-increasing processing unit configured to increase a density of imaging data obtained by scanning the ultrasound beam; and
a display processing unit configured to form a display image based on the imaging data having an increased density,
the density-increasing processing unit, based on depth direction data arranged at a high density along a depth direction of the ultrasound beam within the imaging data, augmenting a density of scanning direction data arranged at a low density in a scanning direction of the ultrasound beam, thereby increasing the density of the imaging data.

2. The ultrasound diagnostic apparatus according to claim 1, wherein
the density-increasing processing unit places a template corresponding to the scanning direction of the ultrasound beam within the imaging data and moves a kernel corresponding to the depth direction of the ultrasound beam for searching for a kernel that matches the template, thereby augmenting a density of the scanning direction data that belongs to the template using the depth direction data that belongs to the kernel which has been found.

3. The ultrasound diagnostic apparatus according to claim 2, wherein,
the density-increasing processing unit searches for a kernel that matches the template by pattern matching between the scanning direction data that belongs to the template and the depth direction data that belongs to the kernel.

4. The ultrasound diagnostic apparatus according to claim 3, wherein
the density-increasing processing unit searches for a kernel that matches the template by pattern matching based on a degree of similarity between the scanning direction data within the template and the depth direction data to be selected from the kernel at a data interval of the scanning direction data.

5. The ultrasound diagnostic apparatus according to claim 2, wherein
the density-increasing processing unit inserts the density-increasing data obtained based on the depth direction data within the kernel that matches the template into a gap of the scanning direction data within the template, thereby increasing the density of the imaging data.

6. The ultrasound diagnostic apparatus according to claim 5, wherein
the density-increasing processing unit assumes a location where the degree of similarity is the best in the gap of the scanning direction data within the template, based on a spatial distribution of the degree of similarity obtained for the search for the kernel that matches the template, and inserts the density-increasing data in the location which is assumed.

7. The ultrasound diagnostic apparatus according to claim 3, wherein
the density-increasing processing unit searches for a plurality of candidate kernels that match the template by pattern matching, and, based on a distance between each of the candidate kernels and the template, selects a kernel that matches the template from among the plurality of candidate kernels.

8. The ultrasound diagnostic apparatus according to claim 3, wherein
the density-increasing processing unit selects a plurality of kernels that match the template, and, based on the depth direction data obtained from the plurality of kernels, obtains the density-increasing data to be inserted in a gap of the scanning direction data within the template.

9. The ultrasound diagnostic apparatus according to claim 8, wherein
the density-increasing processing unit obtains the density-increasing data based on the depth direction data obtained from the plurality of kernels that match the template and a distance between each of the kernels and the template.

10. The ultrasound diagnostic apparatus according to claim 2, wherein
the density-increasing processing unit sets the template and the kernel so as to be of identical size in a real space.

11. The ultrasound diagnostic apparatus according to claim 2, wherein
the density-increasing processing unit, for increasing the density of the imaging data obtained by scanning the ultrasound beam radially or in a sector shape, increases a size of the template in a real space as the depth of a location where the template is to be placed within the imaging data is greater.

12. The ultrasound diagnostic apparatus according to claim 11, wherein
the density-increasing processing unit, for searching for a kernel that matches the template by pattern matching based on a degree of similarity between the scanning direction data within the template and the depth direction data to be selected at a data interval of the scanning direction data from within the kernel, increases the data interval of the depth direction data to be selected from within the kernel as the depth of the location of the template is greater.

13. The ultrasound diagnostic apparatus according to claim 2, wherein
the density-increasing processing unit places templates at a plurality of different locations within the imaging data and searches for a kernel that matches the template at each of the locations, thereby increasing the density of the scanning direction data that belongs to the templates at the plurality of locations.

14. The ultrasound diagnostic apparatus according to claim 13, wherein
the density-increasing processing unit sets the number of the scanning direction data that belong to the template to a fixed value at each of the plurality of locations within the imaging data.

15. The ultrasound diagnostic apparatus according to claim 2, wherein
the density-increasing processing unit, for placing a template at a plurality of different locations within the imaging data and searching for a kernel that matches the template at each of the locations, thereby increasing the density of the scanning direction data that belongs to the template at the plurality of locations, sets a size of the template in a real space to a fixed size at each of the plurality of locations within the imaging data.

16. The ultrasound diagnostic apparatus according to claim 3, wherein
the density-increasing processing unit inserts the density-increasing data obtained based on the depth direction data within the kernel that matches the template into a gap of the scanning direction data within the template, thereby increasing the density of the imaging data.

17. The ultrasound diagnostic apparatus according to claim 5, wherein
the density-increasing processing unit searches for a plurality of candidate kernels that match the template by pattern matching, and, based on a distance between each of the candidate kernels and the template, selects a kernel that matches the template from among the plurality of candidate kernels.

18. The ultrasound diagnostic apparatus according to claim 6, wherein
the density-increasing processing unit searches for a plurality of candidate kernels that match the template by pattern matching, and, based on a distance between each of the candidate kernels and the template, selects a kernel that matches the template from among the plurality of candidate kernels.

19. The ultrasound diagnostic apparatus according to claim 5, wherein
the density-increasing processing unit selects a plurality of kernels that match the template, and, based on the depth direction data obtained from the plurality of kernels, obtains the density-increasing data to be inserted in a gap of the scanning direction data within the template.

20. The ultrasound diagnostic apparatus according to claim 6, wherein
the density-increasing processing unit selects a plurality of kernels that match the template, and, based on the depth direction data obtained from the plurality of kernels, obtains the density-increasing data to be inserted in a gap of the scanning direction data within the template.
